(19) United States
(12) Patent Application Publication
(10) Pub. No.: US 2010/0120034 A1
(43) Pub. Date: May 13, 2010

(54) METHYLATION ANALYSIS OF MATE PAIRS

(75) Inventors: Kevin McKernan, Marblehead, MA (US); Benjamin Schroeder, San Mateo, CA (US); Victoria Boyd, San Carlos, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/498,285

(22) Filed: Jul. 6, 2009

Related U.S. Application Data
(60) Provisional application No. 61/149,976, filed on Feb. 4, 2009.
(60) Provisional application No. 61/133,891, filed on Jul. 3, 2008.

(51) Int. Cl. C12Q 1/68; C07H 21/04

(52) U.S. Cl. 435/6; 536/23.1

(57) ABSTRACT

Various embodiments of the present teachings relate to methods for the methylation analysis of nucleic acids. The subject methods include methods that result in the preparation of mate-pair libraries suitable for highly multiplexed DNA sequencing. Embodiments include methods of preparing mate-pair libraries comprising a first tag sequence and a second tag sequence, wherein one of the tag sequences has been converted by a methylation conversion agent and the other tag sequence has not been converted by the methylation conversion agent. Other embodiments provided include intermediates for making the mate-pair library and kits for making the mate-pair libraries. Also provided is software and computer systems for analyzing the methylation levels of genomic DNA from which the tag sequences were derived.

### PRIORITY CLAIMS

This application claims the benefit of priority to U.S. Provisional Application No. 61/133,891, filed Jul. 3, 2008, entitled, Methylation Analysis of Mate Pairs, and U.S. Provisional Application No. 61/149,976, filed Feb. 4, 2009, entitled, Methylation Analysis of Mate Pairs, which are incorporated herein by reference.

### FIELD

This invention is in the field of analysis of a methylated nucleic acid by means of high throughput nucleic acid sequencing techniques.

### BACKGROUND

Regions of genomic DNA are frequently methylated. The base 5-methyl cytosine is the most frequently encountered methylated base in the DNA derived from eukaryotic cells. 5-methyl cytosine results from methylation of the number 5 carbon in the pyrimidine ring of cytosine. The methylation of genomic DNA, which is reversible, is well-known to have important biological significance. Such areas of biological significance include the activation and inactivation of genomic regions for transcription. For example, carcinogenesis may occur by the methylation of tumor suppressing genes, which may deactivate the genes. Consequently, the analysis of methylation patterns in cancer cells is a major area of research.

Most conventional methods of nucleic acid methylation analysis involve treatment of the nucleic acid of interest with a methylation conversion agent. Exemplary of such conversion agents is sodium bisulfite. Sodium bisulfite converts the nucleic acid base cytosine to uracil. 5-methylcytosine, however, is not converted by sodium bisulfite under conditions employed for methylation analysis. Thus, sequencing the sodium bisulfite-treated DNA will result in the detection of an uracil when the cytosine was not methylated, and the detection of a cytosine when the cytosine was methylated. Many methods exist for manipulating and detecting sequence variations in genomic DNA that has been treated with a methylation conversion agent such as sodium bisulfite. Such techniques include DNA sequencing, real-time PCR, and the oligonucleotide ligation assay (OLA).

There are many methods of high throughput sequence analysis that result in extremely high numbers of relatively short stretches of DNA being sequenced, e.g., the SOLiD™ sequencing system sold by Applied Biosystems or the Genome Analyzer sold by Illumina.

One method of extracting more information from such short DNA sequences is to use mate-pair sequence tags, wherein the approximate distance between the mate-pair sequences on the genome is known. Mate-pairs of sequence tags can be derived from a single polynucleotide fragment. Such genomic fragments used to generate mate-pairs are typically of a length within a pre-determined range of possible lengths, such as, for example 2-3 kb. This length information can be used to help map the sequence information to a genomic reference sequence. Given the relatively short lengths of the sequence reads, such matching back to a reference sequence can be important for assembling accurate sequence information. The use of mate-pair analysis with a methylation conversion agent for methylation analysis can be problematic for mapping back to genomic reference sequences because of reduced sequence complexity after exposure to the methylation conversion. Sequence complexity is reduced because of the loss of cytosines caused by exposure to sodium bisulfite, which results in mate-pairs rich in adenine, thymine, and guanine following amplification.

There is thus a long-felt need in the industry for sequencing methylated DNA quickly and accurately. Methods, reagents, genetic constructs, kits, data analysis systems, and software for addressing the problems associated with reduced sequence complexity arising from the use of methylation conversion agents are provided herein.

### SUMMARY

Various embodiments of the present teachings relate to methods of analyzing the methylation state of genomic DNA. The methods involve fragmenting genomic DNA. In at least one embodiment, the DNA fragments are circularized to produce a double-stranded circular DNA comprising a nick on one strand. A nick translation in the presence of methylation conversion agent resistant nucleotide triphosphate is then performed. The circular genetic construction can be linearized prior to the nick translation reaction. After the nick translation step, two tag regions of a mate-pair are created, wherein the first tag region may comprise methylation conversion resistant nucleotides and the second tag region may lack methylation conversion resistant nucleotides and not be methylation conversion agent resistant. The construction can, in some embodiments, be amplified. The circular genetic construction can in some embodiments comprise a specific binding pair member so as to facilitate strand separation and purification. The tag regions can be sequenced to provide information about the methylation state of the genomic DNA from which the clone was derived.

The present teachings also relate to methods of analyzing the methylation state of genomic DNA comprising fragmenting a genomic DNA and using the fragmented DNA to form linear genetic constructions, each construction having a first tag sequence and a second tag sequence, wherein the first tag and the second tag are derived from a single genomic DNA fragment. In certain embodiments, the first tag sequence may be converted by a methylation conversion agent, while the second tag sequence is not converted by a methylation conversion agent. The constructs can be clonally amplified to provide templates for sequencing.

The present teachings also relate to polynucleotide constructions comprising a first tag sequence and a second tag sequence, wherein the first tag sequence and the second tag sequence are derived from a single fragment of genomic DNA. The first tag may comprise methylation conversion resistant nucleotides that have been incorporated into the construction by an in vitro reaction and, in certain embodiments, the second tag does not comprise incorporated methylation conversion resistant nucleotides. In some embodiments, the genetic construction comprises a specific binding pair member. In some embodiments, the genetic construction comprises primer-binding sites.

Embodiments of the present teachings also include kits comprising an adapter having a first strand having methylation conversion resistant nucleotides and a second strand complementary to the first strand, wherein the second strand optionally comprises methylation conversion resistant nucleotides. Kits can further comprise oligonucleotide primers specific for a strand of the adapter. Kits can also comprise one or more additional reagents for use in carrying out one or more embodiments of the methods disclosed herein, such as a DNA polymerase, a DNA ligase, methylation conversion resistant nucleotides, etc.

The present teachings further relate to methods of matching a DNA sequence to a genomic sequence database, the methods comprising comparing a data record comprising (1) a first tag sequence that corresponds to a DNA sequence that has not been modified by a methylation conversion agent, (2) a second tag sequence that corresponds to a DNA sequence that may have been modified by a methylation conversion agent, and (3) a distance value indicative of the approximate distance in the genome between the first tag sequence and the second tag sequence, with DNA sequence information in the genomic database. Such methods can be implemented by general purpose computers. Embodiments include systems and software for implementing such methods.

Further embodiments of the present teachings relate to methods of amplifying polynucleotides converted by a methylation conversion agent in which primer-adapters may be ligated to fragments of genomic DNA. The adapters may comprise a double-stranded polynucleotide having a first stand and second strand complementary to the first strand, wherein the first strand may comprise methylation conversion resistant nucleotides and, in certain embodiments, the second strand lacks methylation conversion resistant nucleotides. The adapter modified polynucleotide may then be amplified using primers specific for the sequences in the second strand of the adapter, after the sequences have been converted. In at least one embodiment of the present teachings, the first strand may comprise methylation conversion resistant nucleotides and the second strand may optionally lack methylation conversion resistant nucleotides. The second strand of the adapter may optionally be converted into a methylation resistant sequence during a nick translation step with dNTPs comprising 5-methylcystosine (5mC dNTPs), or other methylation conversion resistant nucleotides to generate adapters that are fully methylation conversion resistant on both strands of the DNA. Adapters that are fully methylation conversion resistant on both strands of the DNA will be the same before and after bisulfite conversion.

Embodiments of the present teachings also relate to methods of analyzing the methylation state of a polynucleotide bound to a solid support. In at least one embodiment, the methods involve fragmenting genomic DNA and circularizing a fragment with two cap adapters that create sticky ends and an internal adapter comprising a specific binding moiety. A nick translation may then be performed and the circularized polynucleotide linearized to create two tag regions of a mate-pair. The polynucleotide can be bound to a solid support using a cognate specific binding moiety to bind the specific binding moiety. The double-stranded polynucleotide can be denatured, and the unbound strand may be eluted and collected. One or both of the bound or unbound strands may be exposed to a methylation conversion reagent, such as sodium bisulfite. The converted strand may then be amplified and sequenced to analyze the methylation of the polynucleotide.

### BRIEF DESCRIPTION OF THE DRAWINGS

FIG. 1 is an example of a 2-3 kb fragment of genomic DNA undergoing ligation to add adapters (cap adapters), wherein the cap adapters comprise an EcoP151 restriction endonuclease recognition site;

FIG. 2 shows an adapter modified genomic DNA circularized by sticky end ligation to an internal adapter comprising a biotin on one strand;

FIG. 3 shows the circular DNA construction linearized by incubation with the restriction endonuclease EcoP15I;

FIG. 4 shows the linearized fragment incubated with a nick translation enzyme and the conversion resistant nucleotide 5-methylcytosine (5mC);

FIG. 5 shows the location of the 5mC's in one strand after the nick translation reaction;

FIG. 6 shows the addition of the primer-adapters to the linearized fragment;

FIG. 7 shows the construct in the bottom of FIG. 6 following the removal of the nicks after nick translation;

FIG. 8 shows the selectively recovered strand, i.e., the strand lacking the biotin;

FIG. 9 shows the treatment of the construct with the methylation conversion agent, sodium bisulfite;

FIG. 10 shows the addition of P2 adapters to one end of the bisulfite converted construction containing the two tag regions, wherein PCR is used to fill in the second strand of the P2 region;

FIG. 11 shows the sequence of the internal adapter, the P1-A/P1-B adapter and the P2-A tail;

FIG. 12 shows the sequences of the internal adapter, the 5mC P1-A/P1B adapter, and the P2-A-tailed library amplification primer used in the method illustrated in FIGS. 1-11; and

FIGS. 13-16 show an exemplary method of preparing long mate-pairs using a double-stranded, circularized polynucleotide having a nick on each strand.

### DEFINITIONS AND EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with the embodiments provided herein, the following terms, unless otherwise indicated, shall be understood to have the following meanings:

The term “nucleotide” refers to a phosphate ester of a nucleoside, as a monomer unit or within a nucleic acid. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group can include sulfur substitutions for the various oxygens, e.g. .alpha.-thio-nucleotide 5′-triphosphates. For a review of nucleic acid chemistry, see Shabarova, Z. and Bogdanov, A., Advanced Organic Chemistry of Nucleic Acids, VCH, New York, 1994.

The term “nucleic acid” refers to natural nucleic acids, artificial nucleic acids, analogs thereof, or combinations thereof.

As used herein, the terms “polynucleotide” and “oligonucleotide” are used interchangeably and mean single-stranded and double-stranded polymers of nucleotide monomers (nucleic acids), including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides may have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes deoxythymidine.

Polynucleotides are said to have “5′ ends” and “3′ ends” because mononucleotides react to make oligonucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also can be said to have 5′ and 3′ ends.

The phrases “DNA fragment of interest,” “polynucleotide of interest,” “target polynucleotide,” “DNA template,” “template polynucleotide,” and variations thereof mean the DNA fragment or polynucleotide that one is interested in identifying, characterizing, or manipulating. As used herein, the terms “template” and “polynucleotide of interest” refer to a nucleic acid that is acted upon, such as, for example, a nucleic acid that is to be mixed with polymerase. In some embodiments, the polynucleotide of interest is a double stranded polynucleotide of interest (“DSPI”).

As used herein, the phrases “different strand of a polynucleotide,” “different strand of a nucleic acid molecule,” and variations thereof refer to a nucleic acid strand of a duplex polynucleotide that is not from the same side as another strand of the duplex polynucleotide.

As used herein, the phrase “paired tag,” also referred to as a “tag mate-pair,” “mate-pair,” or “paired-end,” contains two tags (each a nucleic acid sequence) that are from each end region of a polynucleotide of interest. Thus, a paired tag includes sequence fragment information from two parts of a polynucleotide. In some embodiments, this information can be combined with information regarding the polynucleotide's size, such that the separation between the two sequenced fragments is known to at least a first approximation. This information can be used in mapping where the sequence tags came from.

As used herein, the term “nick” refers to a point in a double stranded polynucleotide where there is no phosphodiester bond between adjacent nucleotides of one strand of the polynucleotide.

The term “nick translation” as used herein refers to a coupled polymerization/degradation or strand displacement process that is characterized by a coordinated 5′ to 3′ DNA polymerase activity and a 5′ to 3′ exonuclease activity or 5′ to 3′ strand displacement. As will be appreciated by one of skill in the art, a “nick translation,” as the term is used herein, can occur on a nick or to a gap. As will be appreciated by one of skill in the art, in some embodiments, the “nick translation” of a gap entails the insertion of appropriate nucleotides in order to form a traditional nick that lacks a phosphodiester bond, which is then translated.

As used herein, the phrases “nick is translated into the DNA fragment of interest,” “nick is translated into the polynucleotide of interest,” and variations thereof refer to the translocation of a nick to a position in the strand that includes the nick that is within the DNA fragment or polynucleotide of interest.

An “analog” nucleic acid or nucleotide is a nucleic acid or nucleotide that is not normally found in a host to which it is being added or in a sample that is being tested. The target sequence may not comprise an analog nucleic acid because it is the sequence that is to be identified, modified, or manipulated. Nucleic acid analogs include artificial nucleic acids, synthetic nucleic acids, or combination thereof. Thus, for example, in one embodiment, PNA (peptide nucleic acid) is an analog nucleic acid, as is L-DNA and LNA (locked nucleic acids), iso-C/iso-G, L-RNA, O-methyl RNA, or other such nucleic acids. In at least one embodiment, any modified nucleic acid will be encompassed within the term analog nucleic acid. In other embodiments, an analog nucleic acid can be a nucleic acid that will not substantially hybridize to native nucleic acids in a system, but will hybridize to other analog nucleic acids; thus, in those embodiments, PNA would not be an analog nucleic acid, but L-DNA would be an analog nucleic acid. For example, while L-DNA can hybridize to PNA in an effective manner, L-DNA will not hybridize to D-DNA or D-RNA in a similar effective manner. Thus, nucleotides or nucleic acids that can hybridize to a probe or target sequence but lack at least one natural nucleotide characteristic, such as susceptibility to degradation by nucleases or binding to D-DNA or D-RNA, may be analog nucleotides or nucleic acids in some embodiments. Of course, the analog nucleotide or nucleic acid need not have every difference.

The term “nucleic acid sequencing chemistry” as used herein refers to a type of chemistry and associated methods used to sequence a polynucleotide to produce a sequencing result. A wide variety of sequencing chemistries are known in the art. Examples of various types of sequencing chemistries useful in various embodiments disclosed herein include, but are not limited to, Maxam-Gilbert sequencing, chain termination methods, dye-labeled terminator methods, sequencing using reversible terminators, sequencing of nucleic acid by pyrophosphate detection (“pyrophosphate sequencing” or “pyrosequencing”), and sequencing by ligation. Such sequencing chemistries and corresponding sequencing reagents are described, for example, in U.S. Pat. Nos. 7,057,026; 5,763,594; 5,808,045; 6,232,465; 5,990,300; 5,872,244; 6,613,523; 6,664,079; 5,302,509; 6,255,475; 6,309,836; 6,613,513; 6,841,128; 6,210,891; 6,258,568; 5,750,341; and 6,306,597; and PCT Publication Nos. WO 91/06678 A1; WO 93/05183 A1; WO 06/074351 A2; WO 03/054142 A2; WO 03/004690 A2; WO 07/002,204 A2; WO 06/084132 A2; and WO 06/073504 A2.

As used herein, the term “polymerase chain reaction” (PCR) refers to the method described by K. B. Mullis in U.S. Pat. Nos. 4,683,195 and 4,683,202, which describe a method for increasing the concentration of a segment of a polynucleotide of interest sequence in a mixture of genomic DNA without cloning or purification. This process for amplifying the polynucleotide of interest sequence comprises introducing a large excess of two oligonucleotide primers to the DNA mixture containing the desired polynucleotide of interest sequence, followed by a precise sequence of thermal cycling in the presence of a DNA polymerase. The two primers are complementary to their respective strands of the double stranded polynucleotide of interest sequence. To effect amplification, the mixture is denatured and the primers then annealed to their complementary sequences within the polynucleotide of interest molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest sequence become the predominant sequences (in terms of concentration) in the mixture, they are said to be “PCR amplified”.

“Clonal amplification” refers to the generation of many copies of an individual molecule. Various methods known in the art can be used for clonal amplification. For example, emulsion PCR is one method, and involves isolating individual DNA molecules along with primer-coated beads in aqueous bubbles within an oil phase. A polymerase chain reaction (PCR) then coats each bead with clonal copies of the isolated library molecule and these beads are subsequently immobilized for later sequencing. Emulsion PCR is used in the methods published by Marguilis et al. and Shendure and Porreca et al. (also known as “polony sequencing”). See Margulies, et al. (2005) Nature 437: 376-380; Shendure et al., Science 309 (5741): 1728-1732. Another method for clonal amplification is “bridge PCR,” where fragments are amplified upon primers attached to a solid surface. See, e.g., PCT Publication No. WO 98/44151 and U.S. Pat. No. 6,090,592. These methods, as well as other methods of clonal amplification, both produce many physically isolated locations that each contains many copies derived from a single molecule polynucleotide fragment.

As used herein, “binding moiety” means a molecule that can bind to a purifying moiety under appropriate conditions. The interaction between the binding moiety and purifying moiety is strong enough to allow enrichment and/or purification of the binding moiety and a molecule associated with it, for example, a paired tag clone. Biotin is an example of a binding moiety. In some embodiments, by coupling a binding moiety to an adapter, binding of the binding moiety to a purifying moiety target allows purification of the paired tag clone. In some embodiments, the purifying moiety can be present on a solid support, such as, for example, streptavidin bound to a polystyrene bead.

As used herein, the term “specific binding pair member” means a member of a pair of molecules that specifically bind to one another with sufficient specificity so as to avoid the binding of interfering quantities of background compounds. A “binding moiety” can be a specific binding pair member. A least one member of a specific binding pair, and possibly both members, are biological molecules or analogs thereof, such as proteins, carbohydrates, polynucleotides, metabolic intermediates and the like. Exemplary of such specific binding pairs are biotin and avidin, biotin and streptavidin, lectins and carbohydrates, antibodies and antigens, complementary nucleic acids and nucleic acid analogues. When referring to a pair of specific binding pair members, the second binding pair member can be referred to as the cognate pair member or cognate specific binding pair member. For example, when referring to biotin attached to a nucleic acid, it may be said that the nucleic acid is purified by binding to the cognate specific binding pair member, e.g., avidin. Conversely, biotin could be said to be the cognate specific binding pair member for avidin.

The term “solid support” refers to any solid phase material upon which an oligonucleotide is synthesized, attached, or immobilized. Solid support encompasses terms such as “resin”, “solid phase”, and “support”. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as, for example, glass, silica, controlled-pore-glass (CPG), or reverse-phase silica. The configuration of a solid support can be in the form of beads, spheres, particles, granules, a gel, a surface, or combinations thereof. Surfaces can be planar, substantially planar, or non-planar. Solid supports can be porous or non-porous, and can have swelling or non-swelling characteristics. A solid support can be configured in the form of a well, depression or other container, vessel, feature, location, or position. A plurality of solid supports can be configured in an array at various locations, e.g., positions, addressable for robotic delivery of reagents, or by detection means including scanning by laser illumination and confocal or deflective light gathering.

The term “distance value” means a value indicative of the approximate physical distance in the genome between the first tag sequence and the second tag sequence.

The term “nick translation enzyme” means an enzyme with DNA polymerase activity that also has 5′ to 3′ exonuclease activity, thus giving the appearance of a moving or “translating” a nick (or gap) in a double-stranded region of DNA from one location to another as polymerase and exonuclease activity proceed in concert with one another. Methods for performing nick translation reactions are known to those of skill in the art. See, e.g., Rigby, P. W. et al. (1977), J. Mol. Biol. 113, 237. A variety of suitable polymerases can be used to perform the nick translation reaction, including for example, E. coli DNA polymerase I, Taq DNA polymerase, Vent DNA polymerase, Klenow DNA polymerase I, and phi29 DNA polymerase. Depending on the enzyme used, nick translation can occur by 5′ to 3′ exonuclease activity or by 5′ to 3′ strand displacement.

The term “methylation conversion agent” means a chemical reagent that modifies the chemical structure of a nucleotide base so as to produce a nucleotide base with different base pairing specificity. Exemplary of such reagents is sodium bisulfite (and other bisulfite salts) that deaminates cytosine to produce uracil.

As used herein, the phrases “converted nucleotide,” “converted nucleic acid,” and variations thereof mean any nucleotide base or nucleic acid that has been chemically modified by a methylation conversion agent so as to produce a nucleotide base or nucleic acid with different base pairing. An example of a converted base is the deamination of cytosine to uracil by sodium bisulfite. Thus, cytosine is said to be converted by sodium bisulfite to uracil.

The term “methylation conversion agent resistant nucleotide” means a nucleotide comprising a nucleic acid base that is not chemically altered by the methylation conversion agent (used in a given embodiment) so as to change the base pairing specificity of the nucleotide base. Methylation conversion agent resistant nucleotides are capable of being incorporated by a nick translation enzyme in a primer extension reaction. Exemplary of methylation conversion resistant nucleotides is 5-methylcytosine (5mC) used in conjunction with sodium bisulfite. Thus, 5-methylcytosine is not deaminated when exposed to sodium bisulfite.

The term “adapter” means a synthetic double-stranded polynucleotide. Adapters can be ligated to a polynucleotide so as to facilitate further structural or physical manipulations of the polynucleotide. Adapters can be used to do one or more of the following: introduce amplification primer binding sites, introduce sequencing primer binding sites, introduce restriction endonuclease recognition sites, introduce specific binding pair members, or facilitate the circularization of a linear polynucleotide molecule.

As used herein, the phrase “a full set of dNTPs” means a set of at least 4 nucleotides capable of supporting a nick translation reaction, e.g., dATP, dCTP, dGTP, and dTTP. Various analogs can also be employed in addition to or in place of any one of dATP, dCTP, dGTP, and dTTP, including, but not limited to, methylated bases such as 5-methylcytosine. The phrase “a full set of regular dNTPs” means a set of nucleotides consisting of dATP, dCTP, dGTP, and dTTP.

The terms “tag,” “tag region,” and “tag sequence” as used herein refer to each of the two polynucleotide sections of mate-pair clone that are derived from polynucleotide sequences at the termini of a genomic fragment. Tag regions and tag sequence can be sequenced to produce base pair sequences representative of the actual tag regions. The terms can be used to refer to a sub-sequence of a polynucleotide of interest.

### DESCRIPTION

Various embodiments of the present teachings relate to methods for the methylation analysis of nucleic acids. The subject methods include methods that may result in the preparation of mate-pair libraries suitable for highly multiplexed DNA sequencing. Subject embodiments include methods of preparing mate-pair libraries comprising a first tag sequence and a second tag sequence, wherein one of the tag sequences may be converted by a methylation conversion agent and the other tag sequence may not be converted by the methylation conversion agent. Other embodiments provided include intermediates for making the mate-pair library and kits for making the mate-pair libraries. It also be appreciated that while much of the description provided herein focuses on the use of methylation conversion resistant nucleotides to generate tag regions that are resistant to conversion by methylation conversion agents, the embodiments provided herein can be adapted to take advantage of the inability of many methylation conversion agents to convert nucleotide bases that are base paired, i.e., in double-stranded form.

In various embodiments, genomic DNA obtained from cells of interest is fragmented. Methods of DNA fragmentation and the selection of the proper fragmentation method(s) are well-known to persons of ordinary skill in the art. Such methods include, for example, sonication, shearing, digestion with restriction endonucleases, random chemical degradation, and the like. DNA can be obtained from a variety of different cell types, including both eukaryotic and prokaryotic. DNA can be obtained from a variety of different tissues in higher organisms. In some embodiments, DNA can be obtained from tumors.

In at least one embodiment, the fragmented DNA can be size selected so as to produce a fraction of DNA fragments of the desired size range. Fractionation of DNA fragments according to size is well known to persons of ordinary skill in the art, and such fractionation techniques may include electrophoresis, size exclusion gel chromatography, HPLC, centrifugation, and the like. The use of size fractionated DNA fragments can be used to produce mate-pair libraries in which the approximate distance between the mate-pairs on the genome of interest is known, thereby facilitating matching of the mate-pairs to pre-existing genomic sequence information.

In some embodiments, DNA fragments can be circularized in order to provide for the generation of mate-pair libraries. DNA fragments can be modified so as to enable circularization. Adapters can be added to the ends of the genomic fragments so as to facilitate circularization. Such adapters can be blunt-ended, sticky-ended, or comprise a sticky-end and a blunt-end. After the addition of adapters to the ends of the DNA fragment, the modified fragment can be circularized. Circularization can be achieved by enzymatic or chemical ligation of the ends of the genetic construction to one another or through an intermediate polynucleotide. In some embodiments, the adapter modified fragment can be circularized by ligation to an internal adapter fragment. Internal adapter fragments can optionally comprise a specific binding pair member, e.g., biotin, digoxygenin, and the like.

Internal adapter fragments can be used to facilitate the generation of mate-pair libraries. Internal adapter fragments, in some embodiments, can comprise restriction endonuclease recognition sites for restriction endonucleases that cleave at a site distal to the recognition sequence, e.g., type IIs or type III restriction endonuclease recognition sites. For example, the type IIs or type III restriction recognition sites can be oriented so as to enable the enzyme to cut the genomic DNA in the proximity of the junction between the internal adapter and the genomic DNA so as to generate tag sequences between the cut sites and the junctions. The internal adapter fragments can further comprise a specific binding moiety attached to one strand of the internal adapter. In at least one embodiment, the specific binding moiety is biotin. In some embodiments of the present teachings, the specific binding moiety can be used to remove an undesired strand of a nucleic acid construction in subsequent steps. In other embodiments of the present teachings, the specific binding moiety can be used to isolate a desired strand of a nucleic acid construction. Guidance on the creation of mate-pair libraries can be found in, among other places, PCT Published Application No. WO 05/42781 A2.

In some embodiments of the present teachings, the circular genetic construction formed by circularizing the genomic DNA fragment for analysis will comprise a nick located in one strand of the circular genetic construction. The nick can be located at the junction between the genomic DNA for analysis and an adapter added to the genomic DNA. The nick can be formed by not phosphorylating a 5′ terminus of a strand of the internal adapter, thereby preventing a ligation event from taking place.

After circularization, the circular DNA construction can be linearized so as to produce a genetic construction having a first tag sequence and a second tag sequence at opposite ends of the linear nucleic acid molecule. Generating the tag regions can, in certain embodiments, occur in the same step as the linearization step. In at least one embodiment, the double-stranded cleavage of the circular DNA construction can be achieved by an enzymatic or chemical cleavage. Linearization can be achieved, for example, by making a double-stranded cut in the circular genetic construction in one or more locations. One example of such methods of cleaving the circular genetic constructions is to use a type IIs or type III restriction endonuclease (or equivalents thereof) that is specific for restriction endonuclease recognition sites in the internal adapter.

According to at least one embodiment of the present teachings, the circular genetic construction formed between the genomic DNA fragment of interest and the internal adapter comprises a single-stranded nick. The nick can be subsequently translated during later steps in various embodiments of the present teachings. The nick can be located at the junction between the internal adapter and the genomic DNA fragments, or at a junction between the internal adapter and the adapter-modified genomic fragment. The nick may be located 3′ relative to the tag region that is to remain susceptible to conversion by a methylation conversion reagent. The nick can be created by using an internal adapter that is not phosphorylated at one of its two 5′ termini, thus creating a nick at the desired position during the circularization step. Alternatively, the nick (or nicks if both strands contain a nick) can be introduced by other enzymatic means or chemically, or by a combination of chemical and enzymatic means.

Subsequent to the linearization of the circular genetic construction, the nick can be translated by incubating the genetic construction in the presence of a nick translation enzyme, a suitable buffering environment, and a full set of dNTPs, wherein the set of dNTPs comprises at least one methylation conversion resistant nucleotide. Exemplary of such methylation conversion resistant nucleotides is 5-methylcytosine. In at least one embodiment, one or more of the dNTPs in the full set of dNTPs can be a methylation conversion resistant nucleotide.

During the process of nick translation, DNA synthesis proceeds through only one of the tag sequence regions. The DNA synthesis can, in some embodiments, proceed through the internal adapter region of the linearized construction. In some embodiments, after nick translation, a portion of one strand can comprise methylation conversion resistant nucleotides incorporated during the nick translation reaction. In at least one embodiment, the methylation conversion resistant nucleotides are in one of the tag regions, but not the other. The strand of the linear genetic construction that is not modified by the nick translation enzyme does not comprise the incorporated methylation conversion resistant nucleotides.

According to at least one embodiment, the linear double-stranded genetic constructions that remain after the nick translation reaction can be modified with primer-adapters so as to facilitate manipulation of a strand or strands comprising the tag regions. Primer-adapters can be joined to the linearized genetic construction either before or after treatment of the linearized genetic construction with a methylation conversion agent. In at least one embodiment, the primer-adapters are joined to the linearized genetic construction before treatment with a methylation conversion agent. Primer-adapters can be ligated to the termini of the linear genetic construction. The primer-adapters can comprise a primer binding site for use in amplifications or selective binding to complementary sequences for enrichment of desired products. The primer-adapters do not require 5′ phosphorylated ends, but in some embodiments can have 5′ phosphorylated ends. In at least one embodiment, the ligation product formed between the linearized construction and the primer-adapters can be subjected to a nick translation reaction to remove nicks formed between the 5′ ends of the strands and the primer-adapter and the linearized construction. In at least one embodiment, the nick translation reaction can take place in the absence of methylation conversion resistant nucleotides.

In at least one embodiment, the primer-adapter can contain methylation conversion resistant nucleotides in one strand of a double-stranded adapter used to introduce amplification primer binding sites. As used herein, the primer-adapters containing methylation conversion resistant nucleotides in one strand are referred to as “partially protected primer-adapters.” Partially protected primer adapters can be used to preferentially amplify polynucleotides that have been converted by a methylation conversion agent. The methylation conversion agents, such as sodium bisulfite, do not always completely react with all polynucleotides and nucleic acid bases in a conversion reaction. By having a strand that is converted by the methylation conversion agent and a strand that is resistant to conversion, it is possible to employ complementary oligonucleotide primers specific for the converted primer binding regions of the partially protected primer-adapter so as to enrich or selectively amplify for those polynucleotides that have been converted by the methylation conversion agent. The inventors have discovered that conversion of the nucleotide bases in the primer-adapter by a methylation conversion agent is correlated with conversion of the unprotected bases located in between the primer adapters, e.g., the tag regions and the internal adapters.

After addition of the primer-adapters to the linear genetic construction comprising the tag regions, the strand containing the protected tag regions and the unprotected tag regions can be isolated from the complementary strand, so as to be prepared for subsequent manipulations and analysis, e.g. sequencing. The strands of the linearized genetic construction can be denatured and the desired strand retained. Such purification of the desired member of the denatured polynucleotide strands can be achieved by numerous methods well known to the person of ordinary skill in the art of molecular biology, e.g., electrophoresis, chromatography, and the like. In embodiments employing internal adapters comprising a specific binding pair member, the strand comprising the specific binding pair member may be conveniently separated from the other strand by contacting the specific binding pair member with its cognate specific binding pair member that has been immobilized on a solid support. Examples of such solid supports include glass, plastic, and the like, that are capable of being modified so as to attach the cognate specific binding pair member or moiety to the surface. The free strand in the solution can be easily purified away from the balance trend so as to be available for subsequent manipulations, e.g., sequencing or amplification. In at least one embodiment, the specific binding pair member comprises biotin and its cognate specific binding pair member comprises streptavidin bound to polystyrene beads.

The strand of the linearized genetic construction comprises two tag regions: (1) a first tag region comprising methylation conversion agent resistant nucleotides, and (2) a second tag region that lacks methylation conversion agent resistant nucleotides. In at least one embodiment of the present teachings, the strand of the linearized genetic construction is incubated with at least one methylation conversion agent, such as sodium bisulfite. The use of methylation conversion agents for analysis of DNA is well known to the person skilled in the art. The methylation conversion reaction proceeds as long as necessary to provide reasonable certainty that the majority of accessible unprotected bases are converted. Detailed protocols for the use of bisulfite as a methylation conversion agent can be found, for example, in U.S. Pat. Nos. 7,371,526; 7,368,239; and 7,262,013; and U.S. Patent Application Publication No. US 2006/0286577A. In embodiments employing bisulfite salts as a methylation conversion agent, formamide can be used as a denaturant instead of NaOH, the traditional denaturant for bisulfite methylation analysis.

In at least one embodiment of the present teachings, the methylation conversion reaction can be performed while the linearized genetic construction is bound to a solid support. For example, when the internal adapter comprises biotin as a specific binding moiety, the linearized genetic construction may be bound to streptavidin on a solid support, such as, for example, polystyrene beads. The inventors have discovered that sodium bisulfite conversion can be carried out on bound constructions. In at least one embodiment, the streptavidin polystyrene beads may be non-magnetic. Without wishing to be bound by theory, it is believed that the use of non-magnetic beads may prevent the oxidation of the nucleic acids by the iron present in magnetic beads. It is also believed that converting either the bound or unbound nucleic acid separate from their complement may improve the efficiency of the reaction with sodium bisulfite rendering the nucleic acids fully single stranded. The nucleic acid can be denatured and the unbound nucleic acid collected for subsequent use. In at least one embodiment, the bound nucleic acid, the unbound nucleic acid, or both can be subjected to sodium bisulfite conversion. In embodiments where only one of the bound nucleic acid and the unbound nucleic acid is converted by sodium bisulfite, the unconverted strands can be used as a reference or control sample, as an archive sample, or as another test sample. For example, if the unbound nucleic acid is converted using sodium bisulfite, the bound sample may be kept in its original form for later analysis or testing.

The converted strands exposed to the methylation conversion agent can be amplified prior to DNA sequencing. The standard nucleic amplification technologies such as PCR, rolling circle amplification, whole genome amplification, LCR and the like can be employed. Primer sites located within the primer-adapters can be used as priming sites for PCR and similar primer based amplification techniques. By suitable placement of the primer binding sites, the first tag region and second tag region can be simultaneously amplified in the same amplification reaction. In embodiments employing partially protected primer-adapters, amplification can be achieved using amplification primers specific for primer binding sites that have been converted by the methylation conversion agent, thereby permitting the preferential amplification of nucleic acids that have been converted by the methylation conversion agent. Amplification primers specific for converted primer binding sites can be used to introduce additional primer binding sites. These additional primer binding sites can be used for, among other things, amplification or sequencing.

The converted strands can be used as sequencing templates and may be sequenced using DNA sequencing procedures that are well-known to persons skilled in the art. The methods provided here in produce templates for analysis by a wide variety of DNA sequencing methods. Such methods include traditional DNA sequencing techniques employing in electrophoresis, e.g., Sanger sequencing or Maxim and Gilbert sequencing. The templates produced by the methods provided herein can also be sequenced by so-called “next-generation” sequencing techniques that may be amenable to performing large numbers of sequencing reactions in parallel. Such techniques include pyrosequencing, nanopore sequencing, single base extension using reversible terminators, ligation-based sequencing, single molecule sequencing techniques, and the like, as described in, for example, U.S. Pat. Nos. 7,057,056; 5,763,594; 6,613,513; 6,841,128; and 6,828,100; and PCT Published Application Nos. WO 07/121,489 A2 and WO 06/084132 A2. Many of the next-generation sequencing techniques employ a clonal amplification step, wherein individual template molecules are amplified in such a way as to maintain separate clones during the amplification. Exemplary of such clonal amplification methods are emulsion PCR (ePCR) and solid phase PCR. The use of suitable adapters for the amplification of templates produced by the methods provided herein may facilitate the use of such clonal amplification techniques as preparation of templates for sequencing.

Sequencing of the converted strands containing the first and second tag regions may be performed so as to determine the nucleotide sequence of all or part of both tag regions. The converted tag sequence polynucleotide sequences may be difficult to match to a reference sequence in a genomic database because of the presence of a reduced amount of sequence complexity, e.g., in some samples the converted tag sequence will only have three different nucleotide bases due to the conversion of cytosine to uracil, which base pairs with adenosine and thus reads as thymine. The protected tag sequence can, in some cases, be easier to unambiguously match to a reference sequence in the genomic database because of the greater nucleotide base complexity. As the converted tag region and the protected tag region are part of a mate-pair derived from the same genomic fragment, the approximate physical distance in the genome between the 2 tag regions in the mate-pair is known, and thus can be used to help match the tag regions into the reference sequences and to help provide for the assembly of overlapping regions to produce a larger DNA sequence. Accordingly, in at least one embodiment, the protected tag sequence is matched to a genomic database and then the match may be used as an “anchor” (or location of high certainty) to determine the possible location of the converted tag sequence in the genome based, in part, on the approximate physical distance of the tag regions in the mate-pair so as to find a match for the converted tag sequence. It will be appreciated by those skilled in the art that a match between the nucleotide sequence of the converted tag region and the reference sequence is not necessarily a perfect sequence match, but can take into account some of the changes in nucleotide bases caused by the partial or complete conversion of the bases caused by the methylation conversion agent. Additionally, it will also be understood that a match between the protected tag region and the reference genomic sequence can be other than a match for 100% identity, but can include various SNPs, insertions, deletions, substitutions, and the like. Furthermore, it will be understood that while a given genetic locus can be methylated or unmethylated on a single nucleotide of genomic DNA, preparations of a genomic DNA are derived from multiple cells in a sample, e.g., a tissue sample, and that the some of the genomic DNA can be methylated and some may not be methylated at the same locus within a sample. As noted in U.S. Pat. No. 7,112,404, genomic methylation analysis of genomic DNA in a sample does not necessarily yield a simple choice of methylated vs. unmethylated for a given locus; sometimes, a more quantitative answer is required. By using multiple tag sequences from the same genetic locus, i.e., the same or overlapping converted tag regions, a single base position can be interrogated multiple times so as to produce a composite value indicative of the degree of methylation at a given genetic locus in a sample derived from one or more different cells. For example, a tumor sample can comprise identical regions of DNA, but differing in methylation state between the different cells that are with the tissue sample; sequencing such an aggregate of different cells can give data indicative of methylation state that is neither 100% methylated nor 100% unmethylated at the locus of interest.

Various embodiments of the present teachings also relate to software and computers configured for the implementation of such methods of matching converted tag sequences and protected tag sequences to a database of genomic DNA sequences. The genomic database used comprises genomic data, including in some embodiments the entire genome or genomes of the organism from which the mate-pair library was derived. The nucleotide base sequence information obtained from sequencing the tag regions (or portions thereof) of a mate-pair can conveniently be stored as a data record in a form easily manipulated by an electronic computer. The data record can optionally comprise a value indicative of the approximate physical distance between the tag regions on the genome. However, since in a given genetic library the approximate physical distance between the tag regions may be essentially the same, the physical distance information can be kept as a separate record. The matching of sequence to genomic DNA database can be achieved by using well-known methods of sequence searching algorithms, e.g., BLAST, Smith-Waterman, and the like.

Embodiments of the present teachings can be implemented in digital electronic circuitry, or in computer hardware, firmware, software, or in combinations thereof. Apparatus of the present teachings can be implemented in a computer program product tangibly embodied in a machine-readable storage device for execution by a programmable processor; and method steps of the present teachings can be performed by a programmable processor executing a program of instructions to perform functions of the present teachings by operating on input data and generating output. The present teachings can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs.

Other embodiments of the present teachings include methods for analyzing the methylation state of genomic DNA. These methods may be applied to the mate-pair generation techniques discussed above or used for other forms of methylation analysis that do not involve the creation of mate-pair libraries. One such embodiment includes methods of analyzing the methylation state of genomic DNA in which the genomic DNA is denatured with formamide, rather than sodium hydroxide. Sodium hydroxide is typically used to denature DNA for sodium bisulfite treatment so as to provide for the methylation analysis of DNA. However, strong bases, such as sodium hydroxide, may have unwanted side effects such as depurination of the DNA. The use of formamide as a denaturant has been shown to be effective in permitting bisulfite to efficiently modify genomic DNA for methylation analysis purposes. The use of formamide as a denaturant has also been shown to be effective in permitting bisulfite to efficiently modify genomic DNA obtained from formalin fixed paraffin embedded tissues samples. Formalin fixed paraffin embedded tissues are commonly used to store tissue samples, e.g., as prepared by pathologists.

In at least one embodiment of the present teachings, the methylation state of the genomic DNA sample can be ascertained by mixing the genomic DNA with formamide whereby a mixture is formed. The mixture can then be heated to a temperature sufficient to denature the DNA, and a bisulfite salt, such as, for example, sodium bisulfite, can be added to the mixture so as to allow the bisulfite to react with the free amines on the cytosine in the DNA, thereby sulfonating the DNA. The DNA can then be desulfonated, thereby converting the non-methylated cytosines to uracils.

According to at least one embodiment, the formamide solution employed for denaturation in the subject methods can be in the range of 50 to 100% formamide. The formamide can be in an aqueous solution. In at least one embodiment, the method uses formamide solutions having a concentration of at least 50%, such as at least 75%, at least 90%, or at least 95% formamide.

In at least one embodiment of the present teachings, independent of the use of mate-pair library generation, the DNA for analysis can be present in a gel matrix, such as a polyacrylamide gel. In at least one embodiment, the use of DNA present in a gel matrix may facilitate the ease with which a given technique can be performed and may increase the yield of bisulfite treated DNA because DNA that has been size separated in an electrophoresis separation gel matrix can be bisulfite treated prior to removal of the DNA from the gel matrix. In at least one embodiment, the bisulfite treated DNA can also be amplified in the gel matrix. Amplification may be achieved by a variety of standard nucleic amplification techniques, such as PCR, rolling circle amplification, and the like. Amplification of nucleic acids with gel matrices is well-known to person of ordinary skill in the art and is described, for example, in U.S. Pat. Nos. 6,001,568; 5,958,698; and 5,616,478.

### EXAMPLES

### Example 1

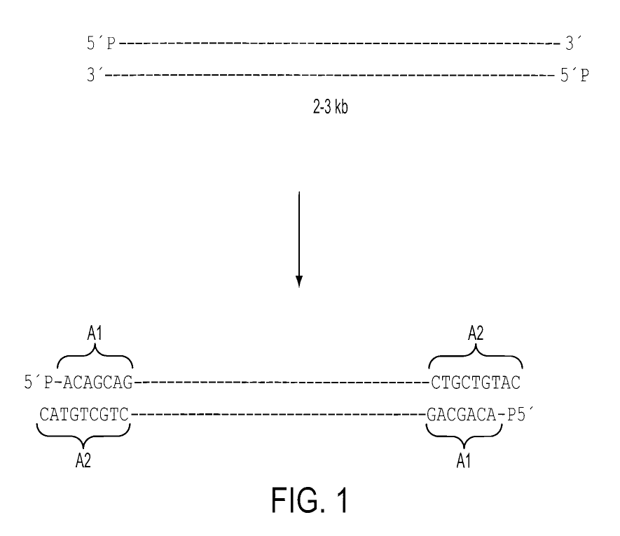

An embodiment of the subject method as applied to the generation of mate-pair libraries for sequencing using the methods described in PCT Published Application No. WO 06/084132 A2, which is herein incorporated by reference for at least the purpose of describing mate-pair library formation and sequencing by ligation with an emulsion PCR preparation step, is provided by way of example. The figures described herein illustrate the preparation and sequencing of a mate-pair library containing clones having first and second tag regions, wherein one of the tag regions has been protected from conversion by bisulfite and is suitable for amplification by emulsion PCR. In the example shown in FIGS. 1-12, the mate-pair library was prepared using EcoP151 cuts, which resulted in short mate-pairs.

FIG. 1 is an example of a 2-3 kb fragment of genomic DNA. In the figure, adapters A1 and A2 are added by ligation. The cap adapters comprise an EcoP151 restriction endonuclease recognition site.

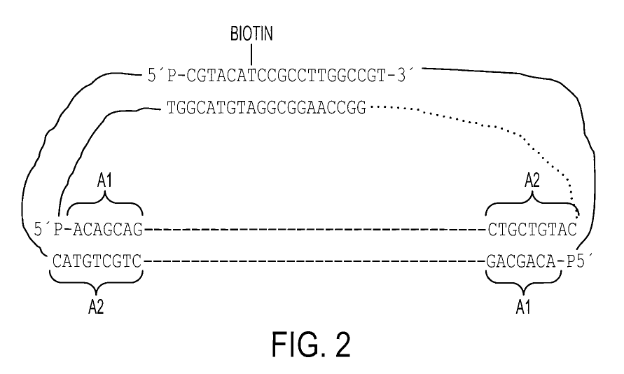

FIG. 2 shows an adapter-modified genomic DNA circularized by ligation to an internal adapter comprising a biotin on one strand. A sticky end ligation was used to join the adapter modified genomic fragment to the internal adapter. The 5′ phosphate on the non-biotinylated strand of the internal adapter was not ligated to the corresponding A2 adapter.

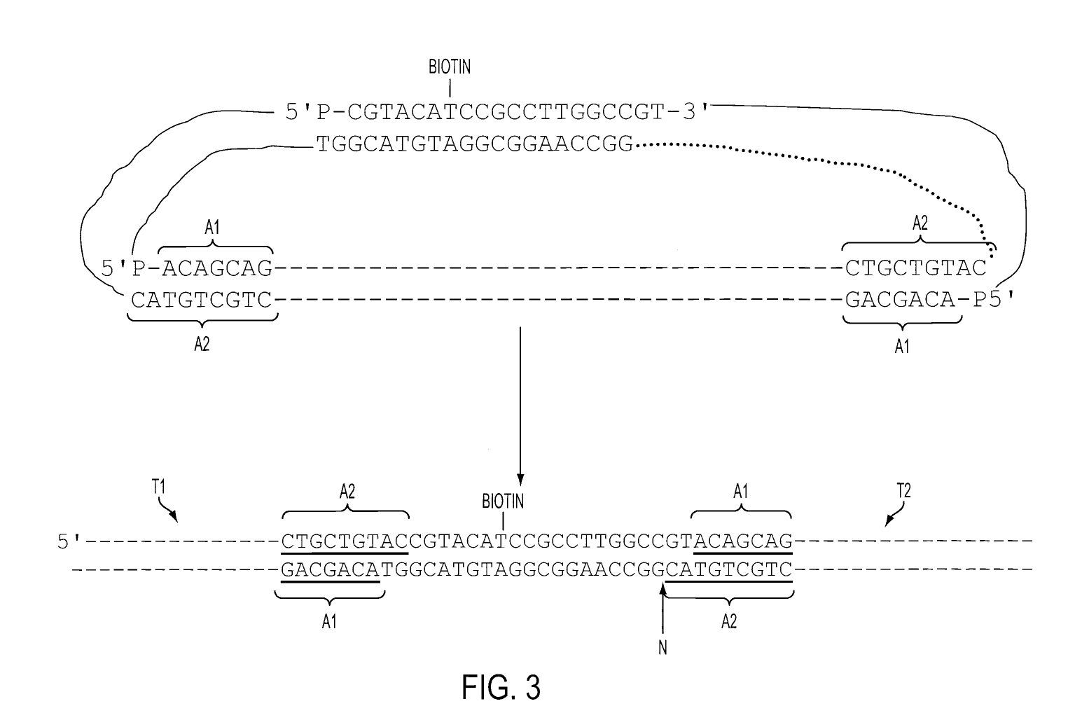

FIG. 3 shows the circular DNA construction linearized by incubation with the restriction endonuclease EcoP151. The nick N in one strand can be seen at the arrow indicating the relative position on the linear genetic construction. Tag regions T1 and T2 are indicated. Tag regions T1 and T2 are approximately 25-27 by each.

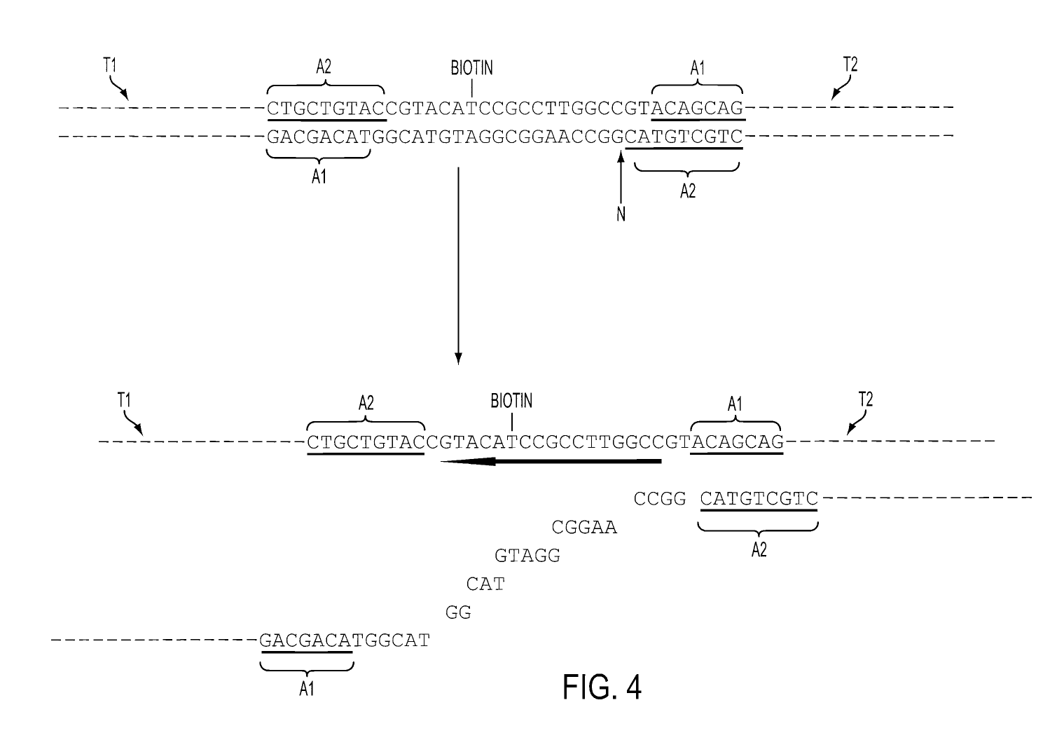

FIG. 4 shows the linearized fragment incubated with a nick translation enzyme and the conversion resistant nucleotide 5-methylcytosine (5mC). Tag T1 also comprises 5mC.

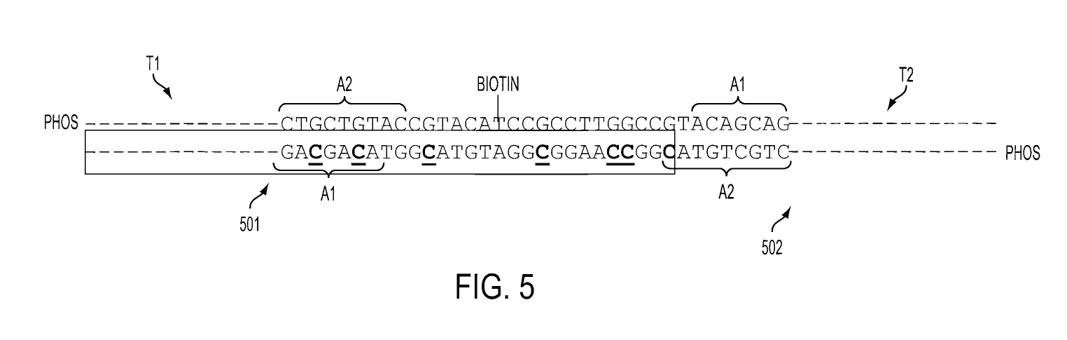

FIG. 5 shows the location of the 5mCs in one strand after the nick translation reaction. The 5mCs in this figure and the following figures are underlined. The box around segment 501 comprises 5mC at all cytosines and preserves the actual genomic sequence resistant to sodium bisulfite. The segment at 502 has native methylation status.

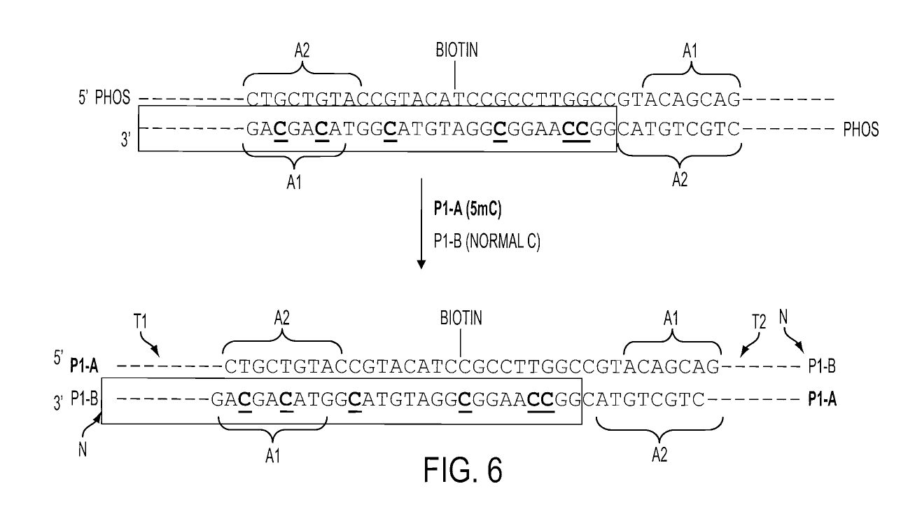

FIG. 6 shows the addition of the primer-adapters P1-A and P1-B (partially protected primer-adapters) to the linearized fragment. The location of nicks N caused by absence of 5′ terminal phosphates on the adapters is also shown.

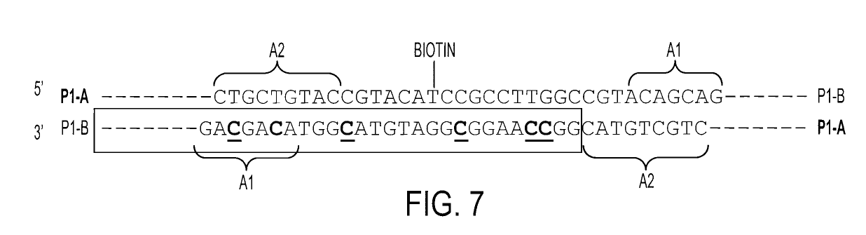

FIG. 7 shows the removal of the nicks after nick translation of the construct shown in the bottom of FIG. 6.

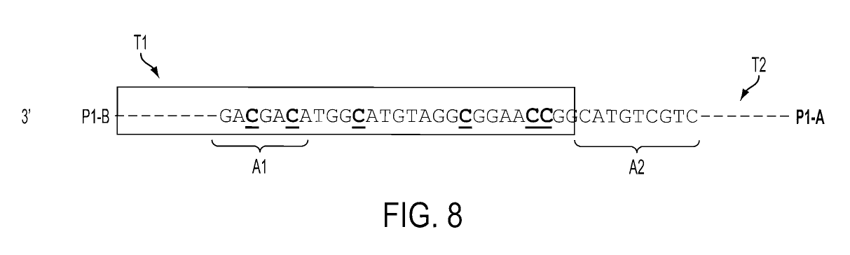

FIG. 8 shows the selectively recovered strand, i.e., the strand lacking the biotin.

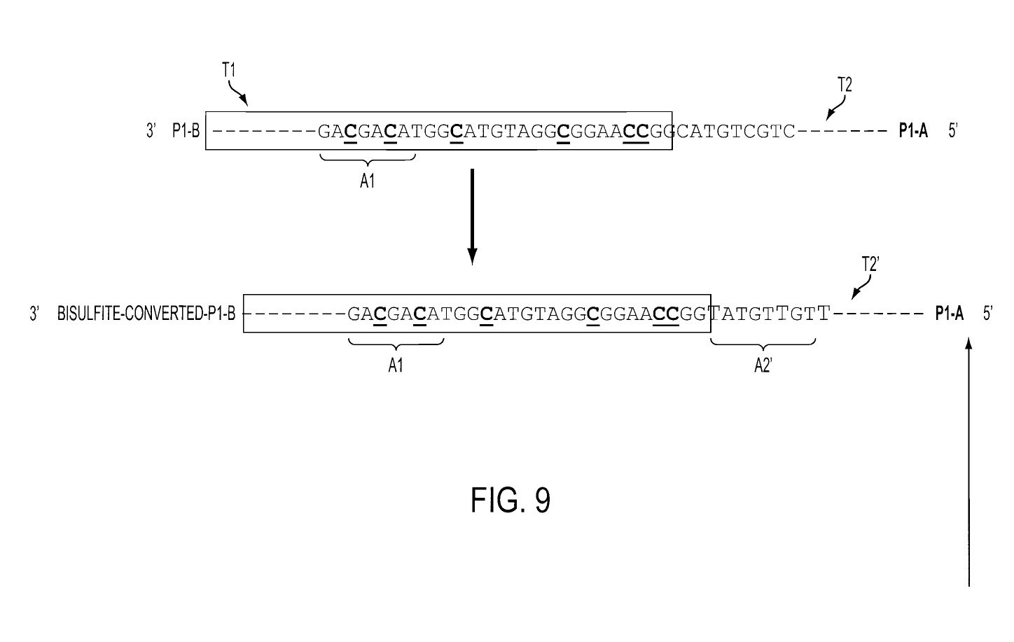

FIG. 9 shows treatment with the methylation conversion agent, sodium bisulfite. P1-B, adapter A2 and tag T2 were converted by bisulfite to produce A2′ and T2′, respectively. The internal adapter, P1-A, and tag T1 were 5mC protected.

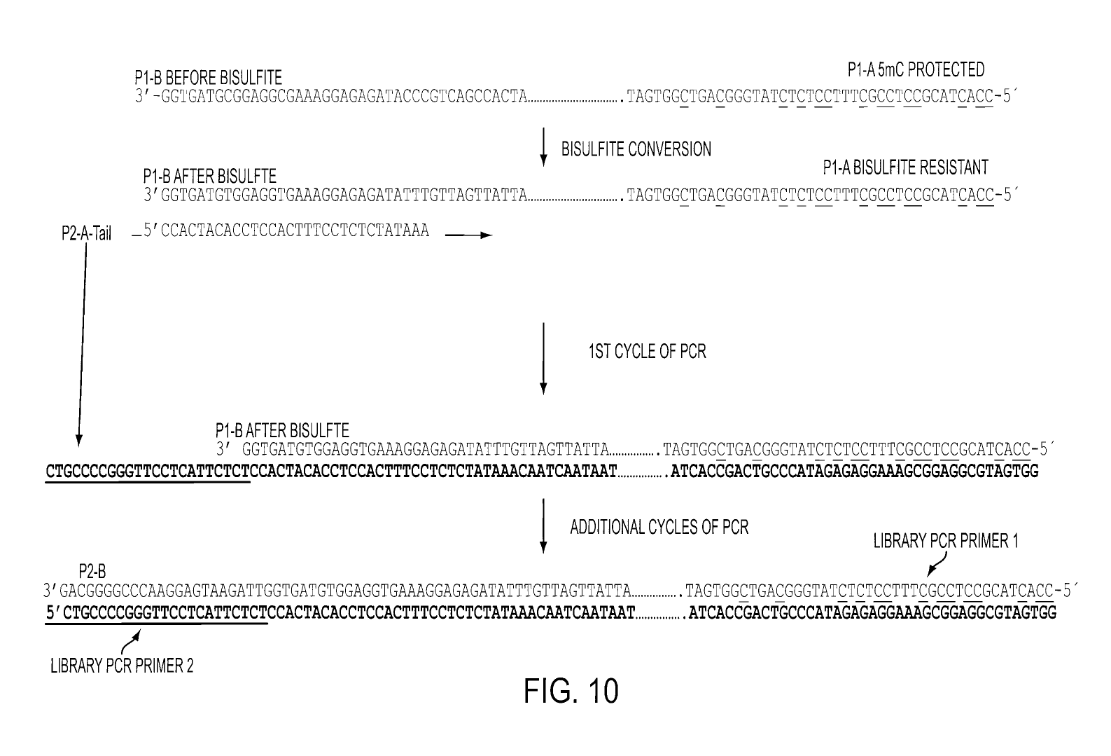

FIG. 10 shows the addition of P2 adapters to one end of the bisulfite converted construction containing the tag regions T1 and T2. PCR was used to fill in the second strand of the P2 region.

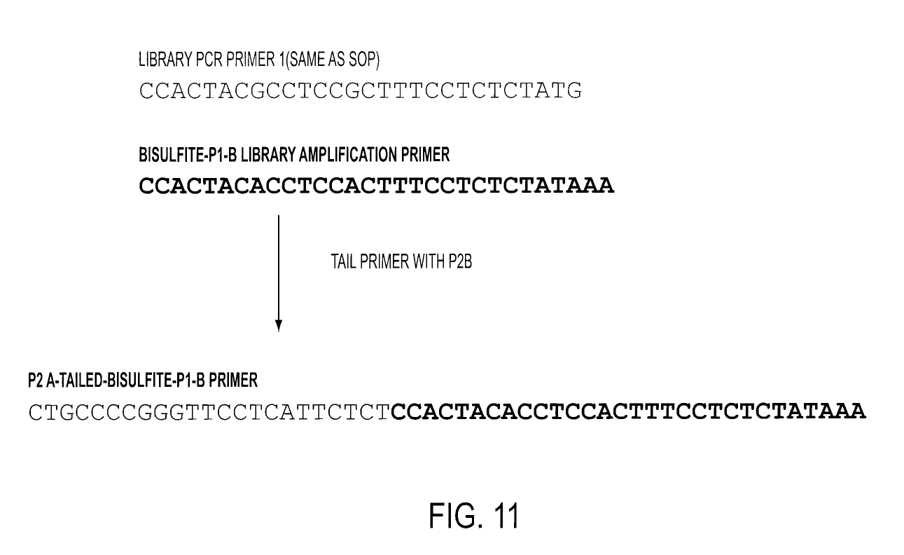

FIG. 11 shows the sequence of the internal adapter, the P1-A/P1-B adapter and the P2-A tail.

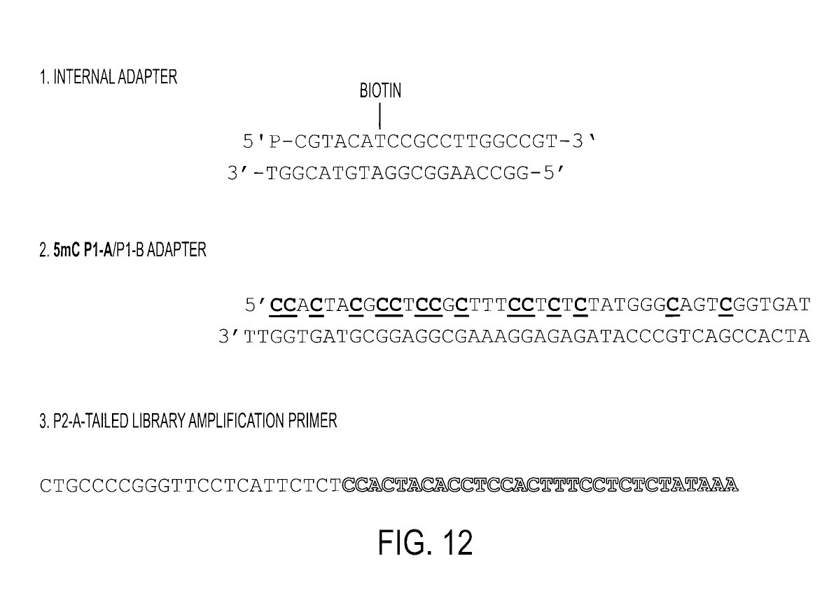

FIG. 12 shows the internal adapter, the 5mC P1-A and P1-B adapters, and the P2-A-Tailed library amplification primer used in the process shown in FIGS. 1-11.

### Example 2

### Mate-Pair Library Generation

### Shearing and End-Repair of the Genomic DNA

1) DNA shearing of 45 ug of E. coli DH10B chromosomal DNA was performed by nebulization in 750 ul of 10 mM Tris pH7.5 as follows:
pressure: 10 psi
time: 2 min 30 sec

### on ice in Nebulizer (Invitrogen)

After nebulization 92% of initial volume was recovered (approx 41 ug DNA, measured by UV absorbance in NanoDrop). 1 ul was analyzed in Bioanalyzer (Agilent) using DNA 7500 Assay. Sheared DNA had a peak at 2, 950 bp:

### 2) DNA Concentration.

DNA was concentrated by ultrafiltration in Nanosep 30K Omega spin cartridge:
Column was loaded with 500 ul of nebulized DNA and spin at 5,000 rcf for 3 min; then the rest was loaded and spun for an additional 4 min. DNA was concentrated to 172 ul (233 ug/ul, UV absorbance, NanoDrop). Thus, 40 ug (98%) of DNA was recovered after ultrafiltration.

### 3) Repair of DNA Ends and Purification of Sample

Repaired and purified as in SOLiD System Mate-Paired Library Preparation, except 13 ul of End-It Enzyme mix (instead of 10 ul) was used to adjust for higher DNA input (40 ug instead of 30 ug). Combined and mixed the following components: Sheared DNA (40 ug)—170 ul; 10× End-It Buffer—30 ul; End-It ATP (10 mM)—30 ul; End-It dNTPs (2.5 mM)—30 ul; Nuclease-free water—27 ul; End-It Enzyme Mix—13 ul

Total: 300 ul. Incubated 30 min at room temperature.
4) Purify the DNA using QIAquick spin columns in the QIAquick Gel Extraction Kit: total of 4 columns were used; DNA was eluted with 25 ul of EB from each column resulting in total of 187 ul of eluate containing 34 ug of DNA.
Methylation of the Genomic DNA EcoP15I Sites: performed as in SOLiD System Mate-Paired Library Preparation except reaction was performed in larger volume to adjust all reaction components to 34 ug DNA input:
1) Methylation reaction:

### Sheared, End-Repaired DNA—187 ul

### 10×NEBuffer 3—35 ul

### 100×BSA—3.5 ul

### EcoP15I Enzyme (10 U/ul) (NEB)—34 ul

S-adenosylmethionine (32 mM)—4.2 ul
Nuclease-free water—86.3 ul

### Total: 350 ul

Incubated at 37° C. for 5 hours
2) Purified the methylated DNA using 4 QIAquick spin columns. After elution with EB buffer, 23.6 ug of DNA was recovered, as measured by UV absorbance (NanoDrop). Ligated the EcoP15I CAP Adapters. Ligated as in SOLiD System Mate-Paired Library Preparation. To ligate CAP adapters to 14.4 pmoles DNA in sample 1440 pmoles of adapter were needed (28.8 ul of 50 pmole/ul CAP stock)
1) Ligation reaction:

### DNA—115 ul

2×NEB Quick ligase buffer—150 ul

### NEB Quick Ligase—8 ul

CAP adapter (ds) (50 pmoles/ul)—28.8 ul

### Total 301.8 ul

Incubated at room temperature for 10 min.
2) Purified DNA using three QIAquick columns, eluted with 30 ul of EB per column. Pooled eluates.
Size-selection of DNA with 1% Agarose Gel
Size-selected as in SOLiD System Mate-Paired Library Preparation. The DNA band of approximately 3 kb (tight size selection) was excised; DNA was extracted from agarose gel using QIAquick Gel Extraction Kit. DNA was eluted from column in 120 ul of EB and analyzed in BioAnalyzer (Agilent) using DNA 7500 Assay:
Mean peak size was found to be at 2845 by (2.8 kb) (see, for example, FIG. 1 above).
DNA concentration was measured by UV absorbance (NanoDrop): 41.7 ng/ul. Thus total 41.7 ng/ul×106 ul=4.42 ug DNA was recovered after this step.

### DNA Circularization

Circularized as in SOLiD System Mate-Paired Library Preparation, except modified internal adapter, NonPhosIA, was used to generate a nick after circularization by ligation. Preparation of the NonPhosIA (SEQUENCE ID NO: 1) which was the same DNA sequence as per the SOLiD protocol, but no 5′P:
Internal adapter, bottom strand without a 5′ P

### NonPhosIAb 5′ GGCCAAGGCGGATGTACGGT (SEQUENCE ID NO: 1)

1. Prepared 1 mM stock of special oligo NonPhosIAb in Low TE buffer.
2. Mixed equal volumes of 1 mM oligonucleotides Top strand normal SOP (biotinylated) internal adapter and NonPhosIAb. Added enough 5× Ligase buffer for a final concentration of 1× Ligase buffer.
Preparation of 200 uL of 50 uM ds-adapter in 1× Invitrogen Ligase buffer Mix:
12.5 uL of the 800 uM biotinylated internal adapter
12.5 uL of the 800 uM modified bottom strand Internal adapter minus a 5′Phos
40 uL of 5× Ligase buffer
135 uL of water
[12.5×10-6×800×10-6=0.00000001 which divided by 200 uL=0.00005 or 50 uM]
3. Hybridized the oligonucleotides by running the following program on a PCR machine:

| Temperature (° C.) | Time (min) |
|---|---|
| 95 | 5 |
| 72 | 5 |
| 60 | 5 |
| 50 | 3 |
| 40 | 3 |
| 30 | 3 |
| 20 | 3 |
| 10 | 3 |
| 4 | forever |

Note: For the 200 uL total volume, it was divided into two equal portions (100 uL) and the above thermalcycling program was followed.
To obtain 95% of circularization efficiency, 4.42 ug of DNA was diluted during circularization reaction to approximately 2.1 ng/ul.
There were 2.34 pmoles of DNA in 4.42 ug of sample of 2.8 kb (0.53 pmoles of DNA/ug×4.42 ug=2.34 pmoles)
Total of 7.02 pmoles of internal adapter were needed (2.34 pmoles×3=7.02), or 3.5 ul of internal adapter stock (2 pmoles/ul).
1) Ligation reaction was set:
DNA (4.4 ug)—106 ul

### 2×NEB Quick Ligase Buffer—1100 ul

NonPhosIA internal adapter (ds) (2 pmoles/ul)—3.5 ul

### Quick Ligase (NEB)—55 ul

Nuclease-free water—935.5 ul

### Total: 2200 ul

Incubated 10 min at room temperature.
2) Purified the DNA using QIAquick column. Eluted 2×30 ul of EB.
3) Treated DNA with Plasmid-Safe ATP-dependent DNase:

### DNA—60 μl

### 25 mM ATP—5 ul

### 10× Plasmid-Safe Buffer—10 ul

### ATP-dependent Plasmid-Safe Dnase (10 U/ul)—1.5 ul

Nuclease-free water—23.5 ul

### Total: 100 ul

Incubated 40 min at 37° C., followed by 20 min at 70° C.
4) Purified DNase treated circularized DNA using QIAquick column. Eluted DNA with 40 ul of EB. Quantitated DNA by UV absorbance (NanoDrop): 7.9 ng/ul. Total: 304 ng of circularized DNA.

### EcoP15I Digestion of Circularized DNA

Digestion as in SOLiD System Mate-Paired Library Preparation, except after EcoP15I digestion step, DNA was cleaned up using ultrafiltration device instead of heat inactivation of enzyme. Heat inactivation was avoided to prevent strand separation, since one of the “circles” of the ds construct was “nicked” due to use of the Non-phosphorylated-internal adapter (NonPhosIA).
1) EcoP15I digestion reaction:
Circularized DNA (304 ng)—38 ul

### 10×NEBuffer 3—10 ul

### 100×BSA—1 ul

### 10 mM Sinefungin—1 ul

### 10×ATP—20 ul

EcoP15I (10 U/ul)—1.5 ul (5 U per 100 ng of 2-6 kb long DNA)
Nuclease-free water—28.5 ul

### Total: 100 ul

Incubated at 37° C. overnight. Then added additional 1 ul 10 mM Sinefungin, 2 ul 10×ATP, and 0.5 ul EcoP16I and continued incubation for additional 1 hour at 37° C.
2) Purified DNA using Microcon 10 ultrafiltration spin device. Reconstituted in 100 ul of NEBuffer 2.

### Nick-translation

1) Assembled on ice the nick-translation reaction:

### DNA in NEBuffer 2—100 ul

5mC-dNTP mix (25 mM each)—1.5 ul
E. coli DNA Polymerase I (10 U/ul)—2 ul

### Incubated 30 min at 16° C.

2) Purified the nick-translated DNA with the Qiagen MinElute Reaction Cleanup kit. Eluted in 40 ul EB. Ligation of partially methylated adapter (SEQUENCE ID NO: 2) (only one adapter was ligated to both ends; adapter has one strand with 5mC). The 5mC positions are underlined:

(SEQUENCE ID NO: 2)

5mC-P1-A (ss): 5′CCA CTA CGC CTC CGC TTT CCT CTC
TAT GGG CAG TCG GTG AT 3′ Length: 41

1. Prepared 800 uM stock of special oligo 5mC-P1-A.
2. Prepared 1 mM (1000 uM) stock of Normal SOP adapter P1-B in Low TE buffer.
Preparation of 200 uL of 50 uM ds-adapter in 1× Invitrogen Ligase buffer

### Mixed:

12.5 uL of the 800 uM 5mC-P1-A

### 12.5 uL of the 1000 uM P1-B

40 uL of 5× Ligase buffer
135 uL of water
[12.5×10-6=×800×10-6=0.00000001 which divided by 200 uL=0.00005 or 50 uM]
3. Hybridized the oligonucleotides by running the following program on a PCR machine:

| Temperature (° C.) | Time (min) |
|---|---|
| 95 | 5 |
| 72 | 5 |
| 60 | 5 |
| 50 | 3 |
| 40 | 3 |
| 30 | 3 |
| 20 | 3 |
| 10 | 3 |
| 4 | forever |

Note: For 200 uL total volume, it was divided into two equal portions (100 uL), and the thermalcycling program was followed.
After EcoP15I digestion, 304 ng of circularized DNA was reduced approximately 29 times. Thus, there were 0.01 ug DNA available for linker ligation. This was 0.01 ug×17.8 pmoles=0.178 pmoles DNA available for ligation. 0.178 pmoles×60=10.68 pmoles adapter needed, or 0.22 ul of 50 uM adapter
1) Ligation reaction:

### Nick-translated DNA—38 ul

5mC-P1-A/P1-B adapter (50 uM)—0.44 ul

### 2× Quick Ligase Buffer—50 ul

### NEB Quick Ligase—2.5 ul

Nuclease-free water—9 ul
Incubated 10 min at room temperature. Purification of library molecules from side products (Streptavidin-Biotin pull out) was performed as in SOLiD System Mate-Paired Library Preparation. Nick-translation of DNA was performed as in SOLiD System Mate-Paired Library Preparation.
1) Nick-translation reaction:
Adapter ligated DNA-Bead complex—37.7 ul
GeneAmp dNTP Blend (100 mM)—0.8 ul

### DNA Polymerase I (10 U/ul)

### Total: 40 ul

### Incubated at 16° C. for 30 min.

2) Washed DNA-Bead complex using magnet in EB. Resuspended DNA-Bead complex in 40 ul EB buffer.

### Removal of Biotinylated Strand and Bisulfite Convertion

The last step of the library preparation before the bisulfite conversion was the capture of the fragments with the biotin on magnetic beads. Only 1-2 ng of fragments was estimated to be present. There were changes to the bisulfite conversion that were used:
Due to the low concentration of DNA for bisulfite conversion, a carrier DNA was spiked into the bisulfite conversion, DH10b, and was not denatured, so it remained double stranded through the bisulfite conversion
The non-biotinylated strand was eluted with base denaturation from the magnetic beads according to the protocol below, immediately prior to bisulfite conversion
Because the non-biotinylated strand was eluted as single stranded, no further steps were needed for denaturation prior to bisulfite conversion—the carrier DNA was deliberately left double stranded
Incubation in bisulfite at 50 degrees for 3 hours was likely sufficient due to short, single stranded fragments of DNA and not large complex genomes with secondary structure.
Microcon 10 as used for the purification to capture the small mate-pair library fragments
   Elution of the non-biotinylated strand for the magnetic beads
   Removed the buffer from the beads. Resuspended the beads in 20 μl of freshly prepared 0.15 M NaOH. Incubated at room temperature for 10 minutes. Put the tube in magnet stand for 1-2 minutes and transferred the supernatant to a new tube. The supernatant contained the non-biotinylated DNA strand. The 20 uL of 0.15 M NaOH solution containing the single-stranded library fragments was mixed with 100 uL of Zymo (reconstituted) CT conversion reagent. One uL of a 300 ng/uL solution of DH10B was added to supply a carrier DNA. No attempt was made to denature the carrier DNA. The reaction was incubated at 50 degrees for 3 hours. The bisulfite reaction was then purified with a Microcon 10 device following the steps below.
   The Microcon 10 washes were as follows:
1. Diluted each bisulfite reaction (if multiples were done) with 100 uL of water. Transferred each diluted reaction to a Microcon 10 and centrifuge at 7000 rpm for 30-40 minutes
2. Removed flow-through and added 100 uL of water to the upper chamber of the M-10 and centrifuge for ˜30 min at 7000 rpm
3. Repeated step 2.
4. Removed flow-through and add 100 uL of 0.1 M NaOH, let sit for 5 minutes at RT, and centrifuged at 7000 rpm for ˜30 min.
5. Removed Flow-through, added 100 uL water, centrifuged for ˜30 Min. at 7000 rpm.
6. Reconstituted the bisulfite converted library in TE (25-50 uL, depending of desired concentration)

### Library Amplification

1) PCR with modified P1 primer

Pre-emulsion Library amplification primer with P2-A tail (SEQUENCE ID NO: 3) P2AtailbisP1 5′

(SEQUENCE ID NO: 3)

CTGCCCCGGGTTCCTCATTCTAACCACTACACCTCCACTTTCCTCTCTAT
AAA

Note: The P2 tail on this Bisulfite-P1 primer sequence (which is the reverse compliment to the bisulfite converted P1B sequence) introduced the P2 sequence recognized by the beads for ePCR according to the SOLiD protocol.
The two primers for library amplification were therefore the “normal” P1 primer and the bisulfite converted P1 primer.
Bisulfite converted library—33 ul
P2A-tailbisP1 primer (50 uM)—1 ul
Library PCR Primer 1 (50 uM)—1 ul

### 10×PCR Gold Buffer w/o Mg++—5 ul

MgCl2 (25 mM)—3 ul
dNTP mix (25 mM each)—0.4 ul

### AmpliTaq Gold (10 U/ul)—2 ul

Nuclease-free water—4.6 ul

### Total: 50 ul

### Thermal Profile:

### 9 min at 95° C.;

95° C. 30 seconds, 55° C. 30 seconds, 70° C. 5 min for 2 cycles

2) Trial-PCR performed as in SOLiD System Mate-Paired Library Preparation

3) Large-scale PCR performed as in SOLiD System Mate-Paired Library Preparation

Large-scale PCR was performed for 40 cycles. DNA was cleaned up with Qiagen MinElute column and eluted with EB buffer

### Example 3

### Fragment Library Preparation

Human gDNA (10 μg) from a male individual of Yoruban ancestry [Coriell cell repository (http://locus.umdnj.edu): NA 18507] was sheared to give fragments (˜60-90 bp) using a Covaris S2 system (Covaris, Woburn, Mass., USA) as described in Chapter 1 of the SOLiD System 2.0 user guide (Applied Biosystems, Foster City, Calif., USA). The sheared DNA was purified with a MinElute Reaction Cleanup kit (Qiagen, Valencia, Calif., USA) as described in the user guide, and then quantified by UV using a NanoDrop ND 1000 Spectrophotometer (Thermo Fisher Scientific, Waltham, Mass., USA). An End-It DNA end-repair kit (Epicentre Biotechnologies, Madison, Wis., USA) was used according to manufacturer instructions to convert DNA with damaged or incompatible 5′- or 3′-protruding ends to 5′-phosphorylated, blunt-end DNA suitable for blunt-end ligation. Following purification of the resultant blunt-end fragments with aforementioned MinElute columns and then quantification by UV, as described above, the required volume of pre-annealed double-stranded adapters needed for ligation was calculated as described in the SOLiD user guide referenced above. The top strand (P1-A) (SEQUENCE ID NO: 4) of the double-stranded P1 adapter was synthesized (TriLink Biotechnologies, San Diego, Calif., USA) with 5mC in place of C to protect the adapter from modification during bisulfite conversion. P1 and P2 adapter sequences were as follows wherein 5mC is underlined.

(SEQUENCE ID NO: 4)

(Top strand) 5mC-P1-A: 5′CCA CTA CGC CTC CGC TTT
CCT CTC TAT GGG CAG TCG GTG AT3′

(SEQUENCE ID NO: 5)

(Bottom strand) P1-B: 3′TT GGT GAT GCG GAG GCG AAA
GGA GAG ATA CCC GTC AGC CAC TA5′

(SEQUENCE ID NO: 6)

(Top strand) P2-A: 3′TCT CTT ACT CCT TGG GCC CCG
TC5′

(SEQUENCE ID NO: 7)

(Bottom strand) P2-B: 5′AGA GAA TGA GGA ACC CGG
GGC AGT T3′

The single-stranded adapter-pairs of oligonucleotides 5mC-P1 and P1-B, and P2-A and P2-B were pre-annealed to form double-stranded adapters. During adapter ligation, only the top adapter strands were joined to the 5′-phosphorylated ends of the DNA fragments. After purification of the ligation products with aforementioned MinElute columns, the bottom adapter sequence was filled-in by extension with DNA polymerase during nick-translation. 2′-deoxycytidine-5′-triphosphate (dCTP) in the conventional mixture of four dNTPs was replaced with 5-methyl-2′-deoxycytidine-5′-triphosphate (5mC-dNTP) (TriLink Biotechnologies). This 5mC-dNTP containing mixture was prepared at 25 mM for each of the four nucleotides using 100 mM stock solutions that included commercially available dNTPs of A, G and T (GE HealthCare-Amersham Biosciences, Pittsburgh, Pa., USA). Following nick-translation, 75 μL of the 80-μL reaction was electrophoresed using a 3% cross-linked agarose gel (Bio-Rad Laboratories, Hercules, Calif., USA) and fragments having the desired size-range (150-200 bp) were excised and then purified with aforementioned MinElute columns. The resultant Yoruban SOLiD fragment-library suitable for bisulfite conversion was quantified by UV as described above, and found to be 12.1 ng/μL or a total yield of 1.21 μg.

### Semi-Quantitative PCR to Monitor Bis-PAGE

Preliminary studies of denaturing DNA embedded in a 6% cross-linked PAGE-slice (see below) compared formamide to NaOH by employing ˜50-ng portions of an Escherichia coli (E. coli) DH10B genomic library) for construction of a SOLiD-60-90 by fragment-library having 5mC-protected ends. The following four conditions were studied: (A.) 25 uL of formamide, (B.) 0.4 M NaOH prepared by us, (C.) NaOH ˜0.4 M supplied as M-Dilution Buffer in the EZ DNA Methylation-Direct kit (Zymo Research) and (D.) ˜0.2M NaOH as M-Dilution Buffer; denaturation with formamide was performed at 95° C. for 5 min. whereas denaturation with NaOH was performed at 37° C. for 15-20 min. Conditions (C.) approximated the commercial kit bisulfite-reaction conditions ignoring the volume of the PAGE-slice whereas condition D approximated the commercial kit bisulfite-reaction conditions taking into account the ˜25-μl volume of the PAGE-slice. Following denaturation, 100 μL of freshly prepared sodium bisulfite obtained as CT Conversion Reagent (Zymo Research, Orange, Calif., USA) was added to each of conditions (A.)-(D.), and the resultant PAGE-slices were incubated for 8 hr at 50° C. Following post-bisulfite washes and desulfonation, each PAGE-slice was subjected to pre-emulsion PCR, all as described below. The number (n) of PCR cycles necessary for an amplicon-band to be visibly detected using FlashGel (Lonza, Basel, Switzerland) was found to be ˜2 less for the library denatured with formamide. This approach was applied to an analogous 5mC-end-protected Yoruban fragment-library at 100-, 10- and 5-ng starting amounts, which gave n=17, 22 and 22, respectively, thus indicating a rough, semi-quantitative, inverse relationship between starting amounts of fragment-library and values of n that appeared to be insensitive to a 2-fold difference between 10- and 5-ng. Despite the limited sensitivity of this approach, it was routinely used for monitoring various pilot experiments including 8 hr vs. overnight incubation with bisulfite at 50° C., which indicated substantial loss of amplifiable fragment-library DNA during overnight conditions.

### Solution Bisulfite Conversion

A 25-μL aliquot containing ˜280 ng of the partially 5mC-end-protected Yoruban SOLiD fragment-library prepared as described above was bisulfite converted according to our reported [Anal Biochem 326 (2004) 278-80.] procedure except for the following modifications. Denaturation was performed by mixing the 25-μL aliquot of the library with 25 μL of highly deionized formamide (Hi-Di Formamide) (Applied Biosystems) and then heating at 95° C. for 5 min. To the resultant solution was added freshly prepared sodium bisulfite obtained as CT Conversion Reagent (Zymo Research), and the reaction mixture was incubated in a 96-well thermal cycler (Applied Biosystems) for 8 hr at 50° C. followed by a programmed hold at 4° C. overnight. A similarly prepared aliquot was incubated overnight for 17 hr at 50° C. Each bisulfite-converted fragment-library was purified as reported [Anal Biochem 326 (2004) 278-80.] except for the following modifications. A Microcon 10 spin-column (Millipore, Billerica, Mass., USA) was used in place of a Microcon 100 spin-column in order to retain the presently described fragment-libraries that are much smaller in size compared to conventionally processed and bisulfite-converted gDNA. In addition, centrifugation speed and time were increased to 7000 rpm and 45 min per wash and for the desulfonation step. Each bisulfite-converted SOLiD fragment-library was recovered in a final volume of 30 μL of sterile buffer (10 mM Tris-HCl, 1.0 mM EDTA, pH 7.2) (Teknova, Hollister, Calif., USA).

### Bis-PAGE Bisulfite Conversion

For comparison of results obtained for solution bisulfite conversion described above, bisulfite conversion was performed directly in a gel-band from PAGE according to the following protocol referred to herein as Bis-PAGE. An aliquot containing ˜100 ng of the final preparation of partially 5mC-end-protected Yoruban SOLiD fragment-library obtained as described above was electrophoresed into a 6% cross-linked DNA Retardation Gel (Invitrogen, Carlsbad, Calif., USA), and the band containing the library was excised using a razor blade. The PAGE slice was then cut into two, approximately equal, halves such that each piece was then small enough to fit into the bottom of a single MicroAmp tube (Applied Biosystems) and be fully immersed upon addition of 25 μL of Hi-Di Formamide (Applied Biosystems). Each ˜50-ng portion of the original fragment-library embedded in the PAGE slice was heated in a 96-well thermal cycler (Applied Biosystems) at 95° C. for 5 min to denature the library fragments followed by cooling to 30° C. to allow addition of 100 μL of freshly prepared CT Conversion Reagent (Zymo Research) and then heating at 50° C. One of these two samples was heated for 8 hr with a programmed hold at 4° C. until the following morning, and the other sample was incubated at 50° C. overnight for 17 hr. Bisulfite reagent was removed by pipet from each Bis-PAGE sample, and then 180 μL of molecular biology-grade water (Sigma, St. Louis, Mo., USA) was added, pipeted up and down several times and then removed. This step was repeated and third wash with fresh water included a 5-min wait before removal, and was repeated in a final, fourth wash. Desulfonation of each embedded Bis-PAGE sample was performed using 180 μL of 0.1 N NaOH that was allowed to stand for 15-20 min before removal. Each still fully intact PAGE slice was then washed twice with 180 μL of water, without a wait step, followed by two washes that each included a 5-min wait time. Each resultant PAGE slice containing embedded bisulfite-converted fragment-library was then immediately used for library amplification prior to emulsion-PCR (pre-emulsion PCR) as described below.

### Library Amplification (Pre-Emulsion PCR)

The following standard P1 and P2 primers were used for SOLiD fragment-library amplification according to the SOLiD System 2.0 user guide (Applied Biosystems).

(SEQUENCE ID NO: 8)

P1: 5′CCA CTA CGC CTC CGC TTT CCT CTC TAT G3′

(SEQUENCE ID NO: 9)

P2: 5′CTG CCC CGG GTT CCT CAT TCT3′

Note that, following bisulfite conversion, double-strand DNA is rendered single stranded and is no longer complementary. Only the strand with bisulfite-resistant ends 5mC-P1-A and 5mC-P2-B is amplified during PCR.

### Amplification of Bisulfite-Converted Libraries in Solution

The master mix specified in the SOLiD System 2.0 user guide (Applied Biosystems) was supplemented as follows with additional AmpliTaq Gold DNA Polymerase to ensure “reading” of U, i.e., deaminated C. For each 1× reaction, 50 μL of Platinum SuperMix (Invitrogen) was mixed with fragment-library PCR primers P1 and P2 (1 μL of 50 μM), 3 μL of the bisulfite-converted DNA (that was recovered as described above in 30 μL of 10 mM Tris-HCl, 1.0 mM EDTA, pH 7.2 sterile buffer) and 0.25 μL of AmpliTaq DNA Polymerase, LD (Applied Biosystems). This 1×PCR reaction was scaled-up 8-fold and dispensed into eight separate tubes to accommodate ˜24 μL of the solution-based bisulfite-converted fragment-library. The 8-hr and overnight bisulfite-conversion samples were processed identically. Thermal cycling as described in the SOLiD System 2.0 user guide (Applied Biosystems) was interrupted periodically (3, 5, 8 and 13 cycles) and 2-μL aliquots of the PCRs were analyzed by FlashGel (Lonza) until amplicon was detected. Thermal cycling was stopped after 13 cycles and PCRs were purified using an AMPure kit (Agencourt, Beverly, Mass., USA) and then quantitatively characterized using a Bioanalyzer 2100 (Agilent, Santa Clara, Calif., USA). A 1-μL aliquot (22 ng or 35 ng for the 8-hr and overnight samples, respectively) was removed for capillary electrophoretic fragment analysis and QC by Sanger sequencing, and the remainder was saved for emulsion-PCR and then SOLiD sequencing.

### Amplification of Bis-PAGE Libraries

Each thoroughly washed and desulfonated Bis-PAGE slice from 8-hr or overnight heating at 50° C. was PCR-amplified in the same MicroAmp tube used for the bisulfite conversion, as described above, using AmpliTaq Gold DNA Polymerase-supplemented conditions identical to those specified in the preceding section on amplification of the bisulfite-converted library in solution. A 2-μL aliquot of each sample was analyzed by FlashGel every other cycle. PCR thermal cycling was stopped after 17 cycles and the concentration of the amplified library was determined using a Bioanalyzer 2100 following purification using an AMPure kit.

Size-Analysis of smPCR Amplicons from Bisulfite-Converted Fragment-Libraries

A ˜1-ng/μL aliquot of each minimally amplified library obtained as described in the preceding sections was serially diluted to give 1-mL of a working solution that was ˜1 copy/μL. The following components were scaled for distribution into multiple 96-well plates for 5-μL PCR: common primers [0.25-μL FAM-short-P1 primer, 0.25-μL normal-P2 primer, 5-μM each; see sequences below incorporating 6-FAM DYE (Applied Biosystems)] were combined with 1.0 μL of the ˜1 copy/μL bisulfite-converted amplified library, 0.5-μL AmpliTaq Gold 10× buffer, 0.4-μL dNTP (2.5 mM each), 0.4-μL MgCl₂ (25 mM), 0.1-μL AmpliTaq Gold DNA Polymerase (5 U/μL), 1.6-μL molecular biology-grade water and 0.5-μL bovine serum albumin-glycerol solution [prepared by mixing 250 μL of a 20 mg/mL bovine serum albumin solution (Sigma, St. Louis, Mo., USA), 700 μL of molecular biology-grade water (Sigma, see above) and 50 μL of Biology-Certified Glycerol (Shelton Scientific-IBI, Peosta, Iowa, USA)]. Thermal cycling conditions were as follows: 5 min at 95° C. (to activate the hot-start polymerase), 40 cycles at 95° C./30 sec, 60° C./2 min, 72° C./45 sec; hold at 4° C.

(SEQUENCE ID NO: 10)

FAM-short-P1: 5′(6-FAM)CGC CTC CGC TTT CCT CTC TAT
G3′

(SEQUENCE ID NO: 11)

normal-P2: 5′CTG CCC CGG GTT CCT CAT TCT3′

A 0.7-μL aliquot of the PCR reaction was added to 11 μL of Hi-Di Formamide (Applied Biosystems) containing 10% ROX 500 size-standard (Applied Biosystems), and heated at 95° C. for 5 min to denature the amplicon. Fragments were analyzed at 60° C. on a 96-capillary 3730×1 DNA Analyzer (Applied Biosystems) using a 50-cm capillary array, POP 7 polymer and GeneMapper Software for data collection with run module GeneMapper50_POP7_{—}1 with dye set Any5Dye (all from Applied Biosystems).

### Sanger Sequencing

In preparation for sequencing, unreacted dNTPs and primers were eliminated by addition of 1 μL of ExoSAP-IT (USB, Cleveland, Ohio, USA) to each PCR sample (after removing the 0.7-μL aliquot for fragment analysis) and incubation at 37° C. for 30 min. This was followed by heat-denaturation at 80° C. for 15 min and then storage at 4° C. The resultant PCR samples were each diluted with 25 μL of water and a 0.5-μL aliquot of the diluted sample was used in BigDye Terminator v1.1 (Applied Biosystems) sequencing by adding 4-μL BigDye Terminator Ready Reaction Mix, 0.5 μL of unlabeled short-P1 primer, 5′CGC CTC CGC TTT CCT CTC TAT-G3′ (SEQUENCE ID NO: 12) (5.0 μM) and 5 μL of water. Cycle sequencing employed 96° C./1 min, followed by 25 cycles of 96° C./10 sec, 50° C./4 min and hold at 4° C. Unincorporated BigDye Terminator and unused primers were removed using the Big Dye XTerminator Purification kit (Applied Biosystems) following manufacturer instructions. Sequencing was performed on a 96-capillary 3730×1 DNA Analyzer (Applied Biosystems)

### Results and Discussion

Representative commercial kits and protocols using DNA-binding matrices for recovery have been shown to afford mostly 4.0-0.5 kb converted-DNA, and could thus lead to substantial loss of bisulfite-converted SOLiD fragment-libraries discussed above. Another concern was the possibly accelerated reannealing (driven by common-adapter sequences) during bisulfite treatment that could prevent complete bisulfite conversion, given the demonstrated requirement for single-stranded regions during the C-sulfonation step.

Nick-translation with 5mC-dNTP was performed in solution, rather than directly in the PAGE gel-slice, in order to better assess completeness of overall C→T conversion that was mentioned above as an acknowledged common source of error in bisulfite-based DNA methylation analyses. The influence of embedding DNA in a PAGE-slice during bisulfite conversion (Bis-PAGE) and subsequent PCR was compared to free-solution reactions in parallel experiments using aliquots of the same SOLiD fragment-library. A 100-ng aliquot of the fragment-library was electrophoresed into a 6% polyacrylamide gel, and the excised PAGE-slice was cut in half so that ˜50-ng portions of the library were bisulfite converted in PAGE (Bis-PAGE) for either 8 hr or 17 hr (“overnight”) at 50° C. Free-solution bisulfite conversion of the same SOLiD fragment-library preparation was performed under each of these reaction conditions using larger, i.e., 240-ng, portions to compensate for expected lower recovery of relatively short fragment-library DNA. Bis-PAGE and free-solution bisulfite treatments bypassed conventional use NaOH to denature DNA by employing formamide, based on recent capillary sequencing results demonstrating that formamide denaturant gave more complete overall C→T conversion compared to NaOH. In this regard, it should be noted a commercially available, highly deionized grade of formamide was used to minimize potential problems due to ionic impurities known to be present in other common grades of formamide. Microcon 10 spin-columns having a lower molecular-weight cutoff range were used in place of previously reported Microcon 100 spin-columns as another means of increasing recovery of relatively short, ˜150-200 by converted DNA library-fragments. Appropriate spin-columns thus bypass use of typical DNA-binding matrices that have been found to provide mostly 4.0-0.5 kb converted-DNA.

Semi-Quantitative PCR Comparison of Denaturation with Formamide vs. NaOH During Bis-PAGE

Preliminary studies of denaturing ˜50-ng of SOLiD fragment-library embedded in a 6% cross-linked PAGE-slice compared formamide at 95° C. for 5 min with either 0.4 M NaOH or 0.2 M NaOH both at 37° C. for 15-20 min. This pre-denaturing was followed by addition of a solution of sodium bisulfite and then incubation at 50° C. for 8 hr. After sequential removal of sodium bisulfite, washing, desulfonation with NaOH and final washing, each PAGE-slice was subjected to PCR. The number (n) of PCR cycles necessary for an amplicon-band to be visibly detected using FlashGel (Lonza, Basel, Switzerland) was found to be ˜2 less for the library denatured with formamide. An inverse relationship between values of n and amounts of starting fragment-library DNA indicates several-fold less PCR-amplifiable DNA in the case of NaOH, which could be due to degradation and/or loss of embedded DNA. Loss of PCR-amplifiable fragment-library DNA was also found for formamide during 50° C. incubation with bisulfite overnight vs. for 8 hr. In this regard, it should be noted that others have previously reported that heating DNA in formamide (without bisulfite) under more forcing conditions (e.g. 110° C., 10 min) than those described herein leads to a low level of cleavage of DNA that was suggested as a chemical sequencing method. In view of this competing side-reaction, any protocol for denaturing and bisulfite conversion of DNA using formamide must avoid excessive heating.

The presently described Bis-PAGE protocol was developed as part of a streamlined sample-prep workflow to enable, for the first time, bisulfite sequencing of genome-wide SOLiD fragment-libraries that will be reported elsewhere. Completeness of overall C→T conversion was unambiguously established by smPCR for capillary sequencing as discussed below. Feasibility studies of extending Bis-PAGE to include conventional gDNA samples was performed. As a representative example, it has been determined that 1 μL containing 50 ng of commercially available (Applied Biosystems) gDNA (CEPH 13470-02) spotted onto a 6% cross-linked PAGE-slice and then air-dried for 5 min could be successfully subjected to the Bis-PAGE protocol described herein for a SOLiD fragment-library. This offers a simplified procedure relative to conventional methods or spin-columns or agarose-embedding using pre-denaturing in NaOH followed by formation of agarose beads in oil.

### Fragment-Library Amplification (Pre-Emulsion PCR)

Comparison of bisulfite-converted SOLiD fragment-libraries involved PCR amplification using a limited number of cycles, as performed for conventional, i.e. non-bisulfite-converted SOLiD fragment-libraries, prior to emulsion-PCR of single molecules for attachment of “clonal” amplicon on beads. During limited amplification of a bisulfite-converted SOLiD fragment-library, the PCR reaction was supplemented with AmpliTaq LD, and the 5mC-protected universal primer-binding site in all members of the library remained unchanged during bisulfite conversion of genomic fragments of interest. Consequently, universal primers for this limited-PCR step amplify library-fragment regardless of whether bisulfite conversion of fragments was complete or not. It was determined to QC bisulfite-treated fragment-libraries derived from either free-solution reaction or Bis-PAGE by measurement of three variables. (1.) Yield was determined by relative recovery, as reflected by semi-quantitative limited PCR, while (2.) sequence and amplicon-size were each accurately determined by established capillary electrophoresis methods. Aliquots of limited-PCR samples were removed at two-cycle intervals for analysis by FlashGel to assess whether an amplicon band could be visually detected. This semi-quantitative discontinuous means of measuring a cycle threshold-like value (“Ct”) akin to real-time PCR Ct-values was estimated to have a sensitivity of roughly ˜2 “Ct” units. Free-solution bisulfite-conversion reactions were distributed into multiple wells at 28 ng of fragment-library/well assuming (for the sake of simplicity) 100% recovery, whereas Bis-PAGE samples (still embedded in PAGE-slices) had ˜50 ng of bisulfite-converted fragment-library DNA assuming (for the sake of simplicity) 100% recovery. A representative well of free-solution fragment-library gave “Ct”=13, whereas the Bis-PAGE fragment-library gave “Ct”=15, which are roughly comparable values considering the assumptions about recovery and the estimated sensitivity of ±2 “Ct” units. In any case, these roughly comparable “Ct” values indicated that loss of short (˜150-200 bp) library-fragments due to diffusion from 6% cross-linked PAGE-slices was insignificant in this first demonstration of Bis-PAGE workflow. Retention of these fragment-libraries was also demonstrated in separate experiments of the type described above starting with smaller amounts of fragment-library, i.e. 10- and 5-ng of input DNA for Bis-PAGE at 50° C. for 8 hr albeit with “Ct”=22, which was consistent with less starting material for PCR. QC of resultant amplicons by capillary methods for size-analysis and sequencing are respectively discussed in the next two sections.

### QC of Single-Molecule Library Fragment Amplicons by Capillary Electrophoretic Size-Analysis

Bisulfite sequencing commonly involves capillary sequencing of bisulfite-converted DNA that has been either cloned to characterize individual molecules or amplified by PCR to characterize ensemble-average molecules. To overcome known sequence-bias during cloning or PCR, and to bypass tedious cloning entirely, recent publications have introduced smPCR for bisulfite sequencing. It was noted in the recent publications that a requirement for successful smPCR is very low occurrence of non-template-dependent amplification commonly referred to as primer-dimer. This problem is exacerbated during smPCR wherein primer concentrations vastly exceed that of a single-molecule in a PCR-well, is not entirely mitigated by use of hot-start reagents, and likely requires optimization of primer sequences. Applicants have found that during troubleshooting bisulfite sequencing that structures of primer-dimers can encompass molecules significantly longer than that of the starting PCR primers. Such primer-dimer related species formed after bisulfite conversion of the presently described fragment-library could therefore be mistaken for actual members of the fragment-library and thus incorrectly indicate incomplete C→T conversion. QC of all smPCRs by capillary electrophoretic sizing of all amplicons that was detected via use of a fluorescently labeled PCR primer, taking advantage of readily available and widely used GeneScan size-standards having a different fluorescent label. These size-standards can therefore be added to all smPCR wells prior to capillary electrophoresis, and interpolated sizes of PCR amplicons precisely calculated by automated GeneMapper software.

The size-range of the SOLiD fragment-library described herein was ˜150-200 bp. Serial dilutions of aliquots of amplified fragment-libraries derived from various reaction conditions were carried out based on UV quantification of the starting amount of DNA in each case. For example, the calculated number of molecules in 1 μL of amplified fragment-library with a starting concentration equal to 2 ng/μL and an assumed ensemble-average fragment-size of 150 by is 1.3×10¹⁰ copies, using an average of 600 g/mole per by for double-stranded DNA. Serially diluting 1 μL into 1 mL provided 13 molecules/μL after 3 of such serial dilutions for further dilutions to in the single-molecule regime for pilot smPCRs (“range-finding”), prior to carrying out a relatively large number of smPCRs to obtain a reasonable Poisson distribution of PCR-wells each having 0 or 1 molecule (or more). A 6-carboxyfluorescein (FAM)-labeled forward (P1) primer was used for smPCR to provide FAM-labeled amplicons for capillary electrophoresis to determine interpolated sizes relative to added rhodamine (ROX)-labeled size-standards. Results confirmed that FAM-labeled amplicons had ˜150-200 bp-sizes as expected for the 5mC-protected SOLiD fragment-library excised following PAGE, and that the number of such FAM-labeled amplicons detected in any given PCR-well decreased with lower concentrations of diluted stock solutions. Such range-finding results generally led to reasonable, Poisson-like single-molecule distributions (see below) that were with ˜two-fold dilution of the ˜1 molecule/μL concentrations calculated as described above. These optimized stock solutions were then used to prepare a total of ˜1,500 5-μL smPCRs in 96-well microtiter plates in batches of 4 plates. Manually processing batches of 4 plates was easily performed on a daily basis and, moreover, was found to mitigate spurious non-template-dependent amplification or primer-dimer problems that occasionally necessitated discarding data plate-wise and repeating smPCRs of such plates.

In some cases, smPCR of a library-fragment gave rise to a group of FAM-labeled peaks, each separated by 1-bp and symmetrically distributed about a major peak that was within the expected range of ˜150-200 bp. This phenomenon was attributed to polymerase slippage at oligo(T) or oligo(A) [or dinucleotide-repeats] regions of DNA during PCR, by analogy to the mechanism originally proposed to explain the observation of “shadow” bands in PCR of DNA having regions of oligo(CA). As has previously been discussed, Sanger-sequencing evidence for slippage at oligo(T) regions having >9 Ts in bisulfite-converted DNA in the context of avoiding such regions when designing PCR primers for amplification and Sanger sequencing. In the presently described SOLiD fragment-library, regions of oligo(T) or oligo(A) with >9 Ts or As within the fragment sequence are, unfortunately, unavoidable due to the random nature of fragment generation and use of universal, fixed-sequence primers for smPCR amplification of all library-fragments. smPCR-wells judged by visual inspection to contain either a single, appropriately sized (FAM-P1/P2)-derived library-fragment in the range of ˜150-200 bp, and those smPCR-wells showing slippage that was not too extensive, were all subjected to Sanger sequencing as described in the next section.

### QC of Single-Molecule Library Fragment Amplicons by Capillary Electrophoretic Sanger Sequencing

Sanger-based sequence analysis of amplicons derived from smPCR of individual library-fragments after confirmatory sizing (see above) established the extent of C→T conversion achieved within each of such library-fragments that is randomly sampled. Sampling a relatively large number of bisulfite-converted library-fragments for this QC analysis thus provides a clear indication of % C→T achieved as a checkpoint for deciding whether or not to proceed with massively parallel, redundant (“deep”) sequencing by means of SOLiD for genome-wide methylome analysis. The extent of genomic coverage achievable by this type of Sanger-sequencing QC analysis of a human genome-wide fragment-library derived from ˜3×10⁹ by gDNA will represent an extremely small percentage of the genome even if many 1000s of library-fragments are randomly sampled by smPCR. On the other hand, even lesser numbers of Sanger-sequenced smPCR amplicons, such as ˜200 discussed below, can provide compelling information on % C→T conversion in view of the following approximations. The ˜150-200 by range of fragments in the library implies an average of ˜175 bases in a single-stranded fragment that has an average C-content of (˜175 bases)×25%=˜44 Cs, excluding for the sake of simplicity 5mCpG dinucleotides and various possible sources of bias. Thus, ˜200 Sanger sequences that each covering an entire fragment provide ˜44 Cs×˜200=˜8,800 Cs that can each be detected as either a C (non-converted) or T (converted). This digital detection and counting therefore represents a dynamic range of nearly 10⁴. In addition, exact sequence-contexts for any non-converted Cs that might be detected could possibly reveal particular sequences wherein Cs resist conversion, especially double-stranded hairpin regions akin to those described in studies of hairpin-bisulfite PCR.

In view of the aforementioned considerations, the Yoruban fragment-library that had been reacted with bisulfite as free-solution DNA or PAGE-slice-embedded DNA (Bis-PAGE) for 8-hr or overnight was serially diluted for smPCR, as discussed above, to provide amplicons for conventional capillary electrophoretic Sanger sequencing. In these initial experiments aimed at comparing the stated reaction conditions, aliquots of optimally diluted sample solutions provided ˜20 smPCRs per 96-well PCR plate. This average smPCR success rate of ˜20% compares favorably with calculated Poisson-distribution percentages of 36% for an average of 1 molecule/well, and 16% for an average of 0.2 molecule/well (or 1 molecule/5 wells). The presently reported design of a SOLiD fragment-library provides for a single orientation after bisulfite conversion such that the forward primer (P1) led to sequencing the strand depleted of C, and the reverse primer (P2) led to sequencing the complementary strand depleted in G. For all four of the reaction conditions specified above, randomly sampled library-fragments leading to smPRC amplicons and corresponding Sanger-sequencing electropherograms were found to be completely converted, i.e. there were no Cs detected other than those present as CpG dinucleotides and thus indicative of 5mCpG dinucleotides in the starting gDNA sample. Careful visual perusal of all of the Sanger-sequencing electropherograms for this preliminary assessment of four different conditions for reaction library-fragments with bisulfite failed to reveal noticeable differences, despite the aforementioned higher “Ct”-like values for samples incubated overnight. Higher “Ct”-like values have been attributed to loss of DNA by acidic and/or other bisulfite-related degradation mechanisms, which have been discussed in detail elsewhere. Alternatively, or in addition, loss of DNA may occur by diffusion of DNA from the PAGE-slice in the case of Bis-PAGE. Degradation mechanisms may have sequence-dependent aspects, and thus represent a possible source of bias that should be minimized in genome-wide bisulfite-sequencing using SOLiD by limiting the C→T conversion processes for fragment-libraries described herein to an 8-hr incubation time. Reducing this and other sources of loss is especially important when starting out with relatively small amounts of gDNA in order to minimize under-representation of sequences in the bisulfite-converted fragment-library that is ultimately subjected to methylome analysis by SOLiD.

To further assess the completeness of bisulfite conversion of the 8-hr Bis-PAGE sample discussed above, ten additional 96-well microtiter plates (960 wells total) containing the optimally diluted Yoruban fragment-library were subjected to smPCR. Instead of applying size-based capillary electrophoretic analysis to select only wells that each contain a single-sequence amplicon, as discussed above, Sanger sequencing reactions were carried out in all 96-wells of each plate (960 wells total) for subsequent capillary electrophoresis. Visual inspection of peak-spacing and peak-color in all of the resultant electropherograms led to identification of ˜200 wells that each contained a single-sequence amplicon. Careful perusal of all of the resultant fragment-sequences revealed the following results. There were two of library-fragments giving rise to Sanger sequences having much longer length, i.e. 190 and 147, compared to other library-fragments, which indicated heterogeneity of shearing and PAGE-sizing during preparation of the library. Furthermore, C was present in all of the ˜200 S anger-sequenced library-fragments almost exclusively in CpG dinucleotides that reflect 5mCpG dinucleotides that were present in the original sample of human, Yoruban gDNA. There were only five other instances of C found to be present at non-CpG sites. Three of these five instances were GpC dinucleotides, which may tentatively be attributed to naturally occurring Gp (5mC) dinucleotides in the original sample of human gDNA.

Common adapter-ends reported herein for ligation to relatively short fragments of gDNA lead to double-stranded SOLiD library-fragments all having the same complementary flanking-sequences. The common complementary flanking sequences represent a significant proportion (up to ˜50%) of the total molecular composition of each library-fragment. In principle, this circumstance could “drive” re-annealing and thus lead to inefficient bisulfite conversion, which is known to require single-stranded regions. This concern proved to be a non-issue by finding >99% conversion of C→T by Bis-PAGE using formamide, based on “gold standard” Sanger sequencing of a relatively large number (˜200) of randomly sampled library-fragments. In addition to the present use of nick-translation directly in a PAGE-slice to streamline construction of this 5mC-protected fragment-library, Bis-PAGE was shown to be a novel means of simplifying sample handling, and reducing the multiplicity of steps, compared to conventional bisulfite conversion of DNA in free-solution. Bis-PAGE provides a way to bypass potential loss of relatively short (˜150-200 base) library-fragments that could likely occur using conventional DNA-binding matrices for recovery. However, prolonged incubation in Bis-PAGE-slices and/or use of insufficiently (<6%) cross-linked polyacrylamide could lead to inadequate recovery and should therefore be avoided. Comparison of Bis-PAGE using formamide for both pre-denaturing and denaturing after addition of bisulfite in place of conventional pre-denaturing with NaOH indicated slightly higher recovery of PCR-amplifiable bisulfite-converted library-fragments with formamide, although the reasons for this are uncertain at the present time. More importantly, limited results of preliminary experiments indicated that human gDNA, without conventional restriction enzyme-mediated cutting to reduce size, could be simply infused into 6% PAGE-slices for successful Bis-PAGE. This offers the possibility of a more convenient bisulfite-conversion protocol applicable to many types of DNA methylation analyses that are available.

### Example 4

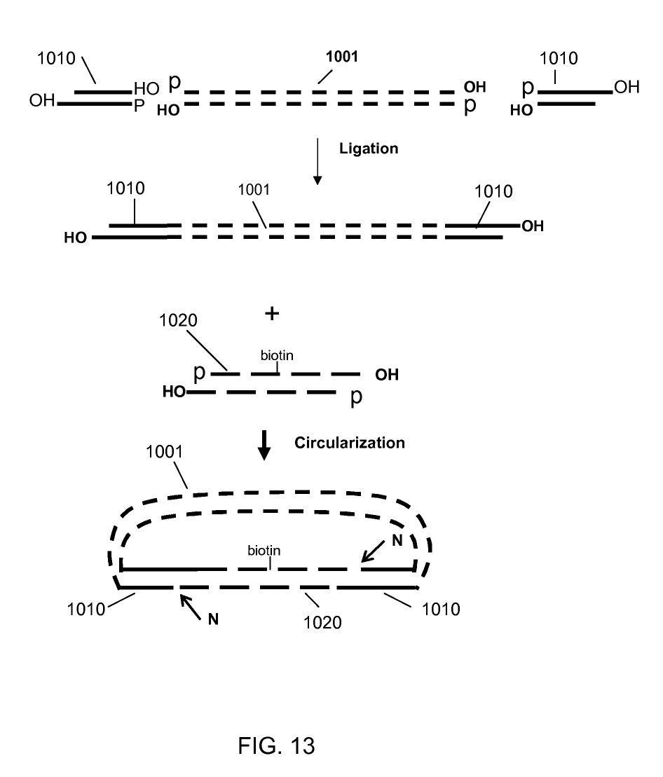

FIGS. 13-16 depict an exemplary method according to the present teachings wherein each of the strands of circularized DNA comprised a nick. The use of a nick on both strands may allow either of the strands to be converted by a bisulfite reaction.

In FIG. 13, cap adapters 1010 were ligated to a DNA fragment 1001. The cap adapters 1010 were missing a 5′ phosphate from one of the oligonucleotides. The missing 5′ phosphate allowed for the formation of nicks N when the DNA fragment 1001 was circularized. A biotinylated internal adapter 1020 was ligated to the cap adapters 1010 to form the circularized polynucleotide.

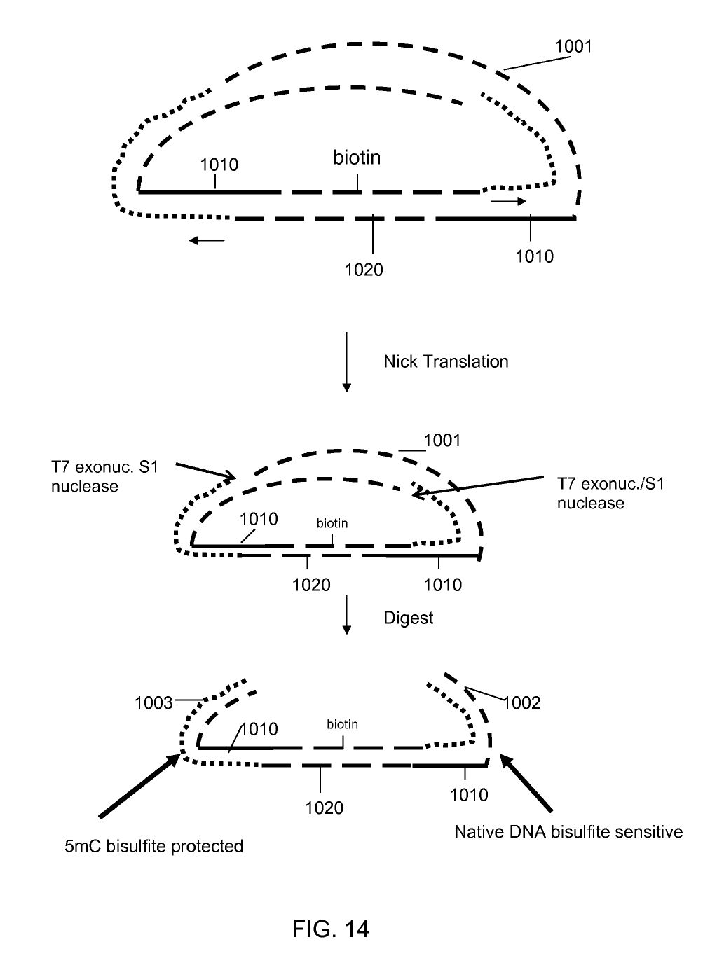

The circularized polynucleotide was nick translated with 5mC dNTP, as shown in FIG. 14. The nick translated polynucleotide was then exposed to T7 exonuclease and S1 nuclease to form long mate-pair tags 1002 and 1003. Due to the use of 5mC dNTP in the nick translation, mate-pair tag 1003 was 5mC bisulfite protected and mate-pair tag 1002 retained its native bisulfite sensitivity.

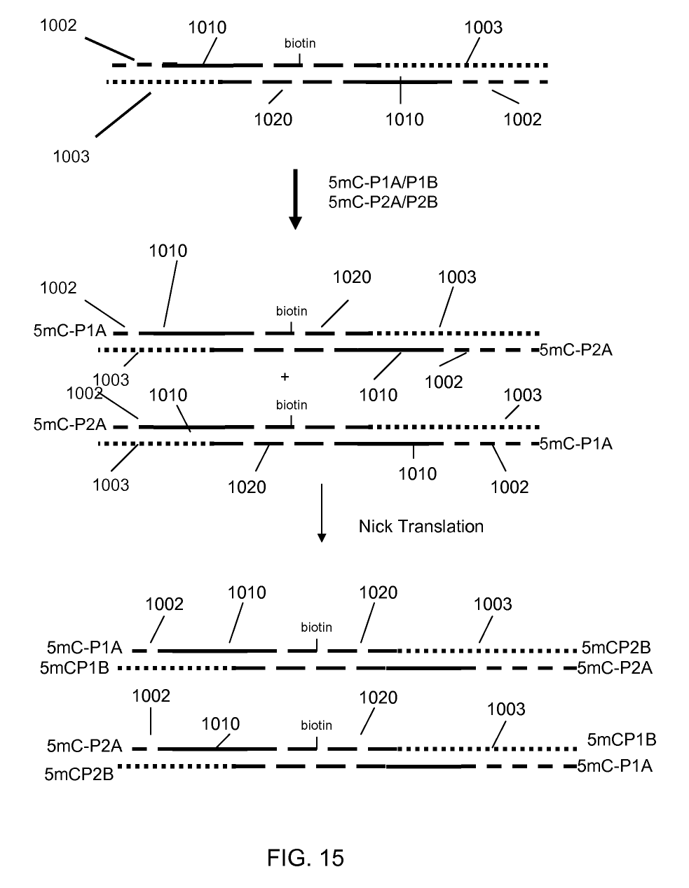

In the first step of FIG. 15, P1 and P2 adapters were ligated to the ends of the DNA. The ligated DNA was then nick translated with DNA polymerase to fill in the non-ligated and non-methyl-C-protected adapter strand.

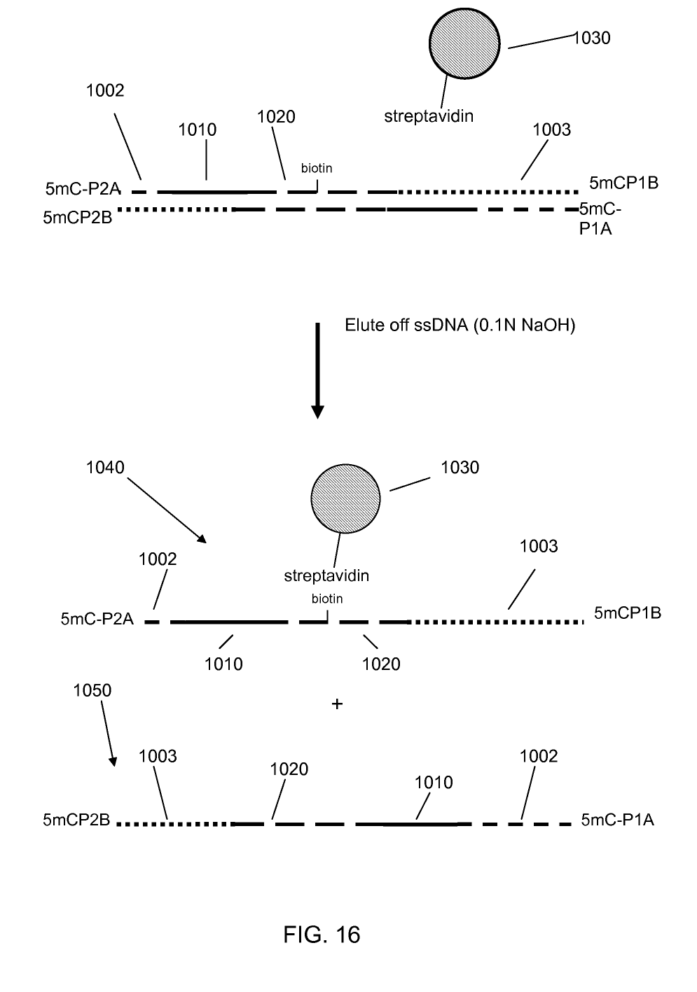

Before bisulfite conversion was carried out, the strands were isolated by capturing the biotinylated strand with streptavidin polystyrene beads 1030. See FIG. 16. The DNA was denatured and the non-captured strand 1050 was separated and eluted off of the captured strand 1040. Once separated, either one or both of strands 1040 and 1050 were ready for bisulfite conversion and subsequent analysis.

### Example 5

The DNA of Example 5 used 90 μg of MCF-7, DNA from a human cancer cell line.

### Shearing the DNA

The genomic DNA was sheared to yield 600 by to 6 kb fragments. To shear for a mate-paired library with insert sizes between 600 by and 1 kb, the Covaris™ S2 system was used. To shear for a mate-paired library with insert sizes between 1 kb and 6 kb, the HydroShear was used. HydroShear used hydrodynamic shearing forces to fragment DNA strands, wherein the DNA in solution flowed through a tube with an abrupt contraction. As it approached the contraction, the fluid accelerated to maintain the volumetric flow rate through the smaller area of the contraction. During this acceleration, drag forces stretched the DNA until it snapped and until the pieces were too short for the shearing forces to break the chemical bonds. The flow rate of the fluid and the size of the contraction determined the final DNA fragment sizes. A calibration run to assess the shearing efficacy of the device prior to starting the first library preparation was performed.

Purification of the DNA with Qiagen QIAquick® Gel Extraction Kit

Sample purification was performed with Qiagen QIAquick® columns supplied in the QIAquick® Gel Extraction Kit. Qiagen QIAquick® columns have a 10-μg capacity, so multiple columns were used during a purification step. For larger amounts of DNA for library construction, phenol-chloroform-isoamyl alcohol extraction and isopropyl alcohol precipitation can be used.

### End-Repairing the DNA

The Epicentre® End-It™ DNA End-Repair Kit was used to convert DNA with damaged or incompatible 5′-protruding and/or 3′-protruding ends to 5′-phosphorylated, blunt-ended DNA for fast and efficient blunt-ended ligation. The conversion to blunt-end DNA was accomplished by exploiting the 5′ [symbol] 3′ polymerase and the 3′ [symbol] 5′ exonuclease activities of T4 DNA Polymerase. T4 polynucleotide kinase and ATP were also included for phosphorylation of the 5′-ends of the blunt-ended DNA for subsequent ligation.

Ligating dsMethyCAP Adapters to the DNA

The ligation of the dsmethyCAP adapter added the methyCAP adapters to both ends of the sheared, end-repaired DNA. The methyCAP adapter was missing a 5′ phosphate from one of its oligonucleotides, which resulted in a nick on each strand when the DNA is circularized in a later step. The dsmethyCAP adapters were included as a 50 uM solution in double-stranded form in the SOLiD™ Mate-Paired Library Bisulfite-Methylation Kit.

### Size-Selecting the DNA

Depending on the desired insert-size range, the ligated, purified DNA was run on a 0.8% or 1% agarose gel. The correctly sized ligation products were excised and purified using the Qiagen QIAquick® Gel Extraction Kit.

### Circularization of the DNA

Sheared DNA ligated to methyCAP Adapters was circularized with a biotinylated internal adapter. To increase the chances that ligation occurred between two ends of one DNA molecule versus two different DNA molecules, a very dilute reaction was used. The circularization reaction products were purified using the QIAquick® Gel Extraction Kit. The biotinylated Internal Adapter dsMethyIA was included as a 2.0 uM solution, double-stranded form in the SOLiD™ Mate-Paired Library Bisulfite Methylation Kit.

Treating the DNA with Plasmid-Safe™ ATP-Dependent DNase

Epicentre® Plasmid-Safe ATP-Dependent DNase was used to eliminate uncircularized DNA. After the Plasmid-Safe™ DNase-treated DNA was purified using the QIAquick® Gel Extraction Kit, the amount of circularized product was quantified. A minimum of 200 ng of circularized product was needed to proceed with library construction. For more complex genomes, 600 ng to 1 μg circularized DNA is needed for a high-complexity library.

Nick-Translating the Circularized DNA with 5mC dNTP-Containing dNTPs

Nick translation using E. coli DNA polymerase I translated the nick into the genomic DNA region. The size of the mate-paired tags produced was controlled by adjusting the reaction temperature and time. The nick translated portion using 5mC was resistant to bisulfite conversion. Therefore, one end of each strand originating from dsDNA genome had a mate-paired portion that bisulfite converted (except for native 5mC bases) and the other Mate-Pair Tag reference matched to the non-bisulfite genome.

Digesting the DNA with T7 Exonuclease and S1 Nuclease

T7 exonuclease recognized the nicks within the circularize DNA and with its 5 [symbol] 3′ exonuclease activity chewed the unligated strand away from the tags creating a gap in the sequence. This gap created an unexposed single-stranded region that was more easily recognized by S1 nuclease and the library molecule was cleaved from the circularized template.

### Capturing on 6.7 Micron Polystyrene Streptavidin Beads Following End-Repair

Regular dNTPs were used for end repair (not 5mC-dNTP) in order to avoid introduction of an inappropriate 5mC in the native strand that would appear to be incomplete bisulfite conversion. The genomic “reference” TAG that was 5mC protected may have occasionally lacked 5mC “protection” because of end-repair, so that a C->T SNP was created. Non-magnetic beads were used to avoid oxidation of the DNA by Fe⁺⁺ during the bisulfite conversion. Capture of the library on polystyrene beads in place of magnetic beads required pelleting the polystyrene by high speed centrifugation in place of using a magnetic stand. By pelleting in the presence of a small percentage of detergent containing buffer (TEX), the beads packed well and the solution above the beads was efficiently removed without disturbing the bead bed. It was safe to leave traces of supernatant on the beads and carry over small amounts from the previous (wash) steps.

### Ligating MethyP1 and MethyP2 Adapters to the DNA

P1 and P2 adapters were ligated to the ends of the end-repaired DNA. The methyP1 and methyP2 adapters were included in double-stranded form as a 50 uM solution in the SOLiD™ Mate-Paired Library Bisulfite Methylation Kit.

Nick-Translating the Library with 5mC dNTP-Containing dNTPs

The ligated, purified DNA underwent nick translation with DNA polymerase. The non-ligated and non-methyl-C-protected adapter strand of the adapter pairs was filled in with 5mC dNTP, fully protecting the adapter sequences during the bisulfite conversion.

### Bisulfite Conversion

The polystyrene beads having double stranded library were attached. Bisulfite conversion required single stranded DNA for efficient bisulfite conversion. The beads were treated with 50 uL of 0.1M NaOH just prior to introduction of bisulfite reagent. The NaOH solution was removed, along with the eluted off single stranded library.

OPTION ONE: It is possible to add the conversion reagent (bisulfite solution) to the beads, incubate at 50° C. for 8 hours. Wash steps and desulfonation may be performed on the library still attached to the polystyrene beads. The beads may then used directly in PCR for library amplification. OPTION TWO: The NaOH solution may also be bisulfite treated and purified with Microcon 100 or PureLink micro PCR kit with a desulfonation buffer for the desulfonation step. Recover bisulfite converted library from column with LoTE.

### Amplification of the Library

The library was amplified using Library PCR Primers 1 and 2 with SOLiD™ Library PCR Master Mix (Platinum Super Mix) supplemented with additional AmpliTaq Gold DNA Polymerase to improve yields in amplification of uracil (from the deaminated cytosine from the bisulfite conversion). In order to achieve whole genome representation during SOLiD sequencing and obtain quantitative accuracy of a human methylome, library amplification did not exceed 17 cycles. Additional cycles may cause PCR-related biases due to differential amplification of library molecules.

### Gel-Purified the Library

The library was run on a 3% agarose gel and the library band (˜300 bp) was excised and eluted using the Qiagen QIAquick® Gel Extraction Kit. The library was then quantified.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

### Example 6

The DNA of Example 6 used 90 μg of MCF-7, DNA from a human cancer cell line.

### Sheared the DNA

### Prepared for Shearing

1. The shearing method used was based on the desired insert size of the mate-paired library (see Table 1).

TABLE 1
Shearing conditions for desired mate-paired library insert sizes.

| Insert Size | | Shearing Method | Shearing Conditions |
|---|---|---|---|
| 600 to 800 | bp | Covaris ™ Shearing in 20% | Number of Cycles: 75 |
| | | glycerol | Bath Temperature: 5° C. |
| | | (13 mm × 65 mm borosilicate | Bath Temperature Limit: 12° C. |
| | | tube) | Mode: Frequency sweeping |
| | | | Water Quality Testing Function: |
| | | | Off |
| | | | Duty cycle: 2% |
| | | | Intensity: 7 |
| | | | Cycles/burst: 200 |
| | | | Time: 10 sec |
| 800 to 1000 | bp | Covaris ™ Shearing in 20% | Number of Cycles: 30 |
| | | glycerol | Bath Temperature: 5° C. |
| | | (13 mm × 65 mm borosilicate | Bath Temperature Limit: 12° C. |
| | | tube) | Mode: Frequency sweeping |
| | | | Water Quality Testing Function: |
| | | | Off |
| | | | Duty cycle: 2% |
| | | | Intensity: 5 |
| | | | Cycles/burst: 200 |
| | | | Time: 10 sec |
| 1 to 2 | kb | HydroShear ® Standard Shearing | SC5 |
| | | Assembly | 20 cycles |
| 2 to 3 | kb | HydroShear ® Standard Shearing | SC9 |
| | | Assembly | 20 cycles |
| 3 to 4 | kb | HydroShear ® Standard Shearing | SC13 |
| | | Assembly | 20 cycles |
| 4 to 5 | kb | HydroShear ® Standard Shearing | SC15 |
| | | Assembly | 5 cycles |
| 5 to 6 | kb | HydroShear ® Standard Shearing | SC16 |
| | | Assembly | 25 cycles |

2. The shearing conditions were tested to ensure that the shearing conditions resulted in the desired insert sizes. Sheared 5 μg DNA and ran 150 ng sheared DNA on a 0.8% E-gel according to the manufacturer's specifications.

### Sheared the DNA Using the Covaris™ S2 System

1. In a round bottom 13 mm×65 mm borosilicate tube, diluted 5 to 20 μg DNA in 500 μL so that the final volume contained 20% glycerol in nuclease-free water.

| Component | Amount | |
|---|---|---|
| 99% Glycerol | 100 | μL |
| DNA | 5 to 20 | μg |
| Nuclease-free water | Variable | |
| Total | 500 | μL |

2. Sheared the DNA using the Covaris™ S2 System shearing program described above.
3. Transfered 500 μL sheared DNA into a clean 1.5-mL LoBind tube.
4. Washed the borosilicate tube with 100 μL nuclease-free water and transferred the wash to the 1.5-mL LoBind tube. Mixed by vortexing and then proceeded to purify the DNA with Qiagen QIAquick® Gel Extraction Kit.
   Purified the DNA with Qiagen QIAquick® Gel Extraction Kit
1. Added 3 volumes Buffer QG and 1 volume isopropyl alcohol to the sheared DNA. If the color of the mixture was orange or violet, added 10 μL 3M sodium acetate, pH 5.5 and mixed. The color turned yellow. The pH required for efficient adsorption of the DNA to the membrane was ≦7.5.
2. Applied 750 μL sheared DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.
13. Quantitated the purified DNA by using 2 μL of the sample on the NanoDrop™ ND-1000 Spectrophotometer (see Appendix B).

### End-Repaired the Sheared DNA

Repairing the Sheared DNA Ends with Epicentre® End-It™ DNA End-Repair Kit

1. Combined and mixed the following components in a LoBind tube.

| Component | Amount |
|---|---|
| Sheared DNA X μg = 15 | 120 μL |
| End-Repair 10X Buffer | 20 μL |
| (Epicentre ® End-It ™) | |
| ATP (10 mM) (Epicentre ® End- | 20 μL |
| It ™) | |
| dNTPs (2.5 mM each) | 20 μL |
| (Epicentre ® End-It ™) | |
| End-Repair Enzyme Mix | 6.7 μL |
| (Epicentre ® End-It ™) | |
| Nuclease-free water (Variable) | 13.3 μL |
| Total | 200 μL |

2. Incubated the mixture at room temperature for 30 minutes.

Purified the DNA with Qiagen QIAquick® Gel Extraction Kit
1. Added 3 volumes Buffer QG and 1 volume isopropyl alcohol to the end-repaired DNA. If the color of the mixture was orange or violet, added 10 μL 3 M sodium acetate, pH 5.5 and mixed. The color turned yellow. The pH required for efficient adsorption of the DNA to the membrane was <7.5.
2. Applied 750 μL end-repaired DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.
13. Quantitated the purified DNA by using 2 μL of the sample on the NanoDrop™ND-1000 Spectrophotometer (see Appendix C).
14. For structural variation studies where tighter size selection of fragments was required, performed one of two size selections (see “Size-select the DNA”) at this point and then proceeded to “Ligate LMP CAP Adapters to the DNA.” If tight insert size distribution were not as critical, proceeded directly to “Ligate LMP CAP Adapters to the DNA.” This optional size-selection was not used if the starting DNA input was less than 10 μg.
   Ligated dsMethyCAP Adapters to the DNA

| | | |
|---|---|---|
| CapBnoPhos | ACAGCAG | (SEQUENCE ID NO: 13) |
| EcoP151 | 5′ PHOS-CTGCTGTAC | (SEQUENCE ID NO: 14) |
| Cap-A (5mC) | | |

### Ligate Thed Adapters to the DNA

1. Calculated the amount of adapter needed for the reaction based on the amount of DNA from the last purification step.

$$For 12 µg of purified end \text{-} repaired DNA with an average$$

$$insert size of 1.5 kb$$

$$\begin{matrix}X pmol \text{/} µg DNA = 1 µg DNA \times \frac{{10}^{6} pg}{1 µg} \times \frac{1 pmol}{660 pg} \times \frac{1}{1500} \\ = 1.0 pmol \text{/} µg DNA\end{matrix}$$

$$\begin{matrix}Y µL adaptor needed = 12 µg DNA \times \frac{1.0 pmol}{1 µg DNA} \times 100 \times \\ \frac{1 µL adaptor needed}{50 pmol} \\ = 24 µL adaptor needed\end{matrix}$$

2. Combined and mixed the components below. If a larger reaction volume was required to incorporate all of the DNA, scaled up the Quick Ligase and Quick Ligase Buffer. Added 1 μL Quick Ligase per 40 μL of reaction volume. Added 1 μL 2× Quick Ligase Buffer per 2 μL of reaction volume.

*From NEB

| Component | Volume (μL) |
|---|---|
| dsMethy-CAP Adapter (ds) (50 pmol/μL) | 22.5 (varied slightly) |
| 2x Quick Ligase Buffer* | 150 |
| Quick Ligase Enzyme* | 7.5 |
| DNA 15 ug | 120 |
| Nuclease-free water | NONE |
| Total | 302 |

3. Incubated the reaction mixture at room temperature for 10 minutes.
   Purified the DNA with Qiagen QIAquick® Gel Extraction Kit
1. Added 3 volumes Buffer QG and 1 volume isopropyl alcohol to the ligated DNA. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL ligated DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. If necessary, pooled the eluted DNA.

### Size-Select the DNA

Size-Selected the DNA Fragments with an Agarose Gel
1. Determined the appropriate percentage of agarose gel needed to size-select DNA.

| Desired Insert Size | | Agarose gel needed (%) |
|---|---|---|
| 600 to 3000 | bp | 1.0 |
| 3 to 6 | kb | 0.8 |

2. Prepared the appropriate percentage agarose gel in 1×TAE buffer with 10 μL of 10 mg/mL ethidium bromide per 100 to 150 mL gel volume. To prepare the 1% gels, used either Agarose-LE (Applied Biosystems, AM9040) or 1% Mini ReadyAgarose Gel (Bio-Rad, 161-3016).
3. Added 10× Gel Loading Solution to the purified ligated DNA (1 μL 10× Gel Loading Solution for every 10 μL DNA).
4. Loaded 1 μL 1 kb DNA ladder. Loaded up to 20 μL dye-mixed sample per well. At least one lane in between the ladder well and the sample wells was used to avoid contamination of the sample with ladder.
5. Ran the gel at 120 V until the marker was close to the edge of the gel.
6. Destained the gel in nuclease-free water twice for 2 minutes each time and visualized the gel on a UV transilluminator with a ruler lying on top.
7. Using the ladder bands and the ruler for reference, excised the band of the gel corresponding to the insert size range of interest with a clean razor blade. If desired, a tighter size selection could be carried out at this stage by taking a tighter cut. If the gel piece was large, it was sliced it up.

### Eluted the DNA Using Qiagen QIAquick® Gel Extraction Kit

1. Weighed the gel slice(s) in a 15-mL polypropylene conical colorless tube.
2. Added 3 volumes Buffer QG to 1 volume of gel.
3. Dissolved the gel slice by vortexing at room temperature until the gel slice was dissolved completely (˜5 minutes).
4. If the color of the mixture was yellow, proceeded to step 5. If the color of the mixture was orange or violet, added 10 μL 3 M sodium acetate, pH 5.5 and mixed. The pH required for efficient adsorption of the DNA to the membrane was ≦7.5.
5. Added one gel volume of isopropyl alcohol to the sample and mixed by inverting the tube several times.
6. Applied about 700 μL sample to the column(s). The maximum amount of gel that could be applied to a QIAquick® column was 400 mg. Used more columns as necessary.
7. Let the column(s) stand for 2 minutes at room temperature.
8. Centrifuged the column(s) at ≧10,000×g (13,000 rpm) for 1 minute and discarded the flow-through.
9. Repeated steps 6 and 8 until the entire sample had been loaded onto the column(s). Placed the QIAquick® column(s) back into the same collection tube.
10. Added 750 μL Buffer PE to wash the column(s).
11. Centrifuged the column(s) at ≧10,000×g (13,000 rpm) for 2 minutes. Discarded the flow-through. Repeated to remove residual wash buffer.
12. Air-dried the column(s) for 2 minutes to evaporate any residual alcohol. Transferred the column(s) to clean 1.5-mL LoBind tube(s).
13. Added 30 μL Buffer EB to the column(s) to elute the DNA and let the column(s) stand for 2 minutes.
14. Centrifuged the column(s) at ≧10,000×g (13,000 rpm) for 1 minute.
15. Repeated steps 13 and 14.
16. If necessary, pooled the eluted DNA in a 1.5-mL LoBind tube.
17. Quantitated the purified DNA by using 2 μL of the sample on the NanoDrop™ND-1000 Spectrophotometer.
   Circularize the DNA with dsMethylInternal Adapter

(SEQUENCE ID NO: 15)

dsMethyIA 5′ (PHOS) CGTACA(BIO-dT)CCGCCTTGGCCGT
3′ TGGCATGT A GGCGGAACCGG-PHOS5′

### Circularized the DNA

1. Prepared a circularization reaction by mixing the components listed below (in order) based on the desired insert size where X was the number of micrograms of DNA to be circularized (see table). If a larger reaction volume was required, scaled up the Quick Ligase and Quick Ligase Buffer. Added 1 μL Quick Ligase per 20 μL of reaction volume.

| | Amount | | | | | | |
|---|---|---|---|---|---|---|---|
| | 600 to | 800 to | 1 to 2 | 2 to 3 | 3 to 4 | 4 to 5 | 5 to 6 |
| Components | 800 bp | 1000 bp | kb | kb | kb | kb | kb |
| Nuclease- | Variable | Variable | Variable | Variable | Variable | Variable | Variable |
| free water | | | | | | | |
| DNA | X μg | X μg | X μg | X μg | X μg | X μg | X μg |
| 2 × Quick | (X × | (X × | (X × | (X × | (X × | (X × | (X × |
| Ligase | 117.5) | 135) μL | 182.5) | 250) μL | 280) μL | 312.5) | 360) μL |
| Buffer | μL | | μL | | | μL | |
| Internal | (X × | (X × | (X × | (X × | (X × | (X × | (X × |
| Adapter (ds) | 3.75) | 2.84) μL | 1.5) μL | 0.9) μL | 0.65) μL | 0.5) μL | 0.4) μL |
| (2 μM) | μL | | | | | | |
| Quick | (X × 6) | (X × | (X × 9) | (X × | (X × 14) | (X × | (X × 18) |
| Ligase | μL | 6.75) μL | μL | 12.5) μL | μL | 15.6) μL | μL |
| (Use | | | | | | | |
| double) | | | | | | | |
| Total | (X × | (X × | (X × | (X × | (X × | (X × | (X × |
| | 235) μL | 270) μL | 365) μL | 500) μL | 560) μL | 625) μL | 720) μL |

For DNA in 2 to 3 kb range circularized

| Components | Amount |
|---|---|
| Nuclease-free | 552.3 μL |
| water | |
| Variable | |
| DNA 3.0 μg | 120 μL |
| 2x Quick | 750 μL |
| Ligase Buffer | |
| dsMethyIA | 2.7 μL (varied |
| Internal | slightly with the |
| Adapter (ds) | measured amount |
| (2 μM) | of DNA is the 7 |
| | samples.) |
| Quick Ligase | 75 μL |
| (2X) | |
| Total | 1500 μL |

2. Incubated at room temperature for 10 minutes.
   Purified the DNA with Qiagen QIAquick® Gel Extraction Kit
1. Added 3 volumes Buffer QG and 1 volume isopropyl alcohol to the circularized DNA. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL circularized DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.

### Isolate the Circularized DNA

Treated the DNA with Plasmid-Safe™ ATP-Dependent DNase

1. Combined and mixed the components below.

For 3.46 μg×6 of DNA used in the circularization reaction.

| Components | Volume (μL) | |
|---|---|---|
| ATP (25 mM) | 5 | |
| 10x Plasmid-Safe ™ Buffer | 10 | |
| Plasmid-Safe ™ DNase (10 U/μL) | 1.15 | μL |
| DNA (3.46 μg) | 60 | μL |
| Nuclease-free water | 24 | μL |
| Total | 100 | μL |

2. Incubated the reaction mixture at 37° C. for 40 minutes.

Purified the DNA with Qiagen QIAquick® Gel Extraction Kit
1. Added 3 volumes Buffer QG and 1 volume isopropyl alcohol to the Plasmid-Safe™ DNase-treated DNA. If the color of the mixture was orange or violet, added 10 μL 3 M sodium acetate, pH 5.5 and mixed. The color turned yellow. The pH required for efficient adsorption of the DNA to the membrane was <7.5.
2. Applied 750 μL Plasmid-Safe™ DNase-treated DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.
13. Quantitated the purified DNA by using 2 μL of the sample on the NanoDrop™ ND-1000 Spectrophotometer (see Appendix C).
   Nick-Translate the Circularized DNA with 5mC Containing dNTPs (25 mM Each)

### Nick-Translated the Circularized DNA

1. This step created the 5mC bisulfite protected tags. Combined and mixed the components listed below on ice. First, mixed all of the components except the enzyme and chilled on ice. Added the enzyme, quickly vortexed and immediately proceeded to the next step.

For 1 μg of Circularized DNA

| Components | Amount | |
|---|---|---|
| dNTP Mix (100 mM, 25 mM | 5 | μL |
| each) | | |
| 10x NEBuffer 2 | 50 | μL |
| DNA Polymerase I (10 U/μL) | 10 | μL |
| DNA 1000 ng | 60 | μL |
| Nuclease-free water | 375 | |
| VARIABLE | | |
| Total | 500 | μL |

2. Incubated the reaction at 0° C. in an ice-water bath for 12 to 14 minutes.
3. Stopped the reaction immediately by proceeding to “Purify the DNA with Qiagen QIAquick® Gel Extraction Kit.”
   Purify the DNA with Qiagen QIAquick® Gel Extraction Kit
1. Added 3 volumes Buffer QG and 1 volume isopropyl alcohol to the nick-translated DNA. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL nick-translated DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.
   Digest the DNA with T7 Exonuclease and S1 Nuclease
   Digested the DNA with T7 Exonuclease
1. Combined:

For 1.26 μg of circularized DNA in each of the 4 samples:

| Component | Amount |
|---|---|
| DNA 1260 ng Always 60 μL | 60 μL |
| NEBuffer 4, 10x | 63.2 μL |
| T7 exonuclease (10 U/μL) | 25.3 μL |
| Nuclease-free water Variable | 483.5 μL |
| Total | 632 μL |

2. Incubated the reaction mixture at 37° C. for 30 minutes. Immediately proceeded to the next step.
   Purified the DNA with Qiagen QIAquick® Gel Extraction Kit
1. Added 3 volumes Buffer QG and 1 volume isopropyl alcohol to the T7 exonuclease digested DNA. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL T7 exonuclease digested DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s).
Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.
   Digested the DNA with S1 Nuclease
1. Freshly diluted Invitrogen S1 nuclease to 1 U/μL with S1 dilution buffer.
2. Combined:
   For T7 exonuclease digested DNA from 1260 ng circularized DNA for each of the 4 tubes in the previous step (The total amount of DNA prior to linearization was 5.056 μg divided into the 4 tubes based on the circularized DNA present. The actual μg of DNA was much less after it has been linearized):

| Component | Amount |
|---|---|
| T7 exonuclease digested DNA | 60 μL |
| 1260 ng | |
| S1 nuclease buffer, 10x | 63.2 μL |
| 3 M sodium chloride | 31.6 μL |
| 100 mM magnesium chloride | 63.2 μL |
| S1 nuclease, diluted to 1 U/μL | 25.3 μL |
| Nuclease-free water Variable | 388.7 μL |
| Total | 632 μL |

3. Incubated the reaction mixture at 37° C. for 30 minutes. Immediately proceeded to the next step.
   Purified the DNA with Qiagen QIAquick® Gel Extraction Kit
1. Added 3 volumes Buffer QG and 1 volume isopropyl alcohol to the digested DNA. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL digested DNA in Buffer QG to the column(s). The maximum amount of DNA that could be applied to a QIAquick® column was 10 μg. Used more columns as necessary.
3. Let the column(s) stand for 2 minutes at room temperature.
4. Centrifuged the column(s) at ≧10,000×g (13,000 rpm) for 1 minute and discarded the flow-through.
5. Repeated steps 2 and 4 until the entire sample had been loaded onto the column(s). Placed the QIAquick® column(s) back into the same collection tube.
6. Added 750 μL Buffer PE to wash the column(s).
7. Centrifuged the column(s) at ≧10,000×g (13,000 rpm) for 2 minutes. Discarded the flow-through. Repeated to remove residual wash buffer.
8. Air-dried the column(s) for 2 minutes to evaporate any residual alcohol. Transferred the column(s) to clean 1.5-mL LoBind tube(s).
9. Added 30 μL Buffer EB to the column(s) to elute the DNA and let the column(s) stand for 2 minutes.
10. Centrifuged the column(s) at ≧10,000×g (13,000 rpm) for 1 minute.
11. Repeated steps 9 and 10.
12. If necessary, pooled the eluted DNA.

### End-Repair the Digested DNA

The end-repaired DNA was repaired with a regular dNTP mix comprising no 5mCdNTP. During SOLiD sequencing, the 5mC preserved sequence may have had a T where there was an end-repaired C. Because most Cs are not methylated, use of “regular” dNTPs erred on the side of an occasional missed 5mC.

Repaired the Digested DNA Ends with the Epicentre® End-It™ DNA End-Repair Kit

1. Prepared Streptavidin Binding Buffer:

| Components | Volume (μL) |
|---|---|
| 500 mM Tris-HCl (pH 7.5) | 10 |
| 5 M Sodium chloride | 200 |
| 500 mM EDTA | 1 |
| Nuclease-free water | 289 |
| Total | 500 |

2. Combined:

| Component | Amount | |
|---|---|---|
| S1 digested DNA X ng | 60 | μL |
| End-repair buffer, 10X | 10 | μL |
| 10 mM ATP | 10 | μL |
| Regular dNTPs (2.5 mM each) | 10 | μL |
| End-Repair Enzyme Mix* | 2 | μL |
| Nuclease-free water Variable | 8 | |
| Total | 100 | μL |

*From the Epicentre ® End-It ™ DNA End-Repair Kit

3. Incubated the reaction mixture at room temperature for 30 minutes.

4. Stopped the reaction by combining and mixing the components below:

| Components | Volume (μL) |
|---|---|
| First End-repaired DNA | 100 |
| 500 mM EDTA | 5 |
| Streptavidin Binding Buffer | 200 |
| Second End-repaired DNA | 100 |
| Total | 405 |

### Bind the Library Molecules to POLYSTYRENE-Streptavidin Beads

### Pre-Washed the Beads

1. Prepared 1×BSA:

| Components | Volume (μL) |
|---|---|
| 100x BSA | 5 |
| Nuclease-free water | 495 |
| Total | 500 |

2. Vortexed a 5 mL bottle of Spherotech streptavidin beads (6.7 micron beads supplied as a 5% w/vol slurry in water) to thoroughly suspend the polystyrene beads in solution. Transferred 200 μL per library sample (1 mg of beads/200 μL) into a 1.5-mL LoBind Tube using a 1 mL pipette tip with a suitable pipettor.
3. Centrifuged at ≧10,000×g (13,000 rpm) for 1 minute. Discarded the supernatant without disturbing the polystyrene bed.
4. Added 400 μL×Bead Wash Buffer and vortexed for 15 seconds. Afterwards, pulse-spun, and added 100 μL1×TEX Buffer, briefly vortexed, and centrifuged at ≧10,000×g (13,000 rpm) for 1 minute.
5. Discarded the supernatant without disturbing the polystyrene bead bed.
6. Added 400 μL1×BSA and vortexed for 15 seconds. Afterwards, pulse-spun, and added 100 μL1×TEX Buffer, briefly vortexed, and centrifuged at ≧10,000×g (13,000 rpm) for 1 minute.
7. Discarded the supernatant without disturbing the polystyrene bead bed.
8. Added 400 μL1× Bind & Wash Buffer and vortexed for 15 seconds. Afterwards, pulse-spun, and added 100 μL1×TEX Buffer, briefly vortexed, and centrifuged at ≧10,000×g (13,000 rpm) for 1 minute.
9. Discarded the supernatant without disturbing the polystyrene bead bed.

### Bound the Library DNA Molecules to the Beads

1. Added the entire 405 μL solution of library DNA in Streptavidin Binding Buffer to the pre-washed beads and vortexed.
2. Mixed by rotation at room temperature for 30 minutes. Afterwards, pulse-spun.

### Washed the Bead-DNA Complex

1. Prepared 1× Quick Ligase Buffer:

| Components | Volume (μL) |
|---|---|
| Quick Ligase Buffer, 2× | 300 |
| Nuclease-free water | 300 |
| Total | 600 |

2. Added 100 μL1×TEX Buffer to the library-bead attachment reaction, briefly vortexed, and centrifuged at ≧10,000×g (13,000 rpm) for 1 minute.
3. Discarded the supernatant without disturbing the polystyrene bead bed.
4. Added 400 μL1× Bead Wash Buffer and vortexed for 15 seconds. Afterwards, pulse-spun, and added 100 μL1×TEX Buffer, briefly vortexed, and centrifuged at ≧10,000×g (13,000 rpm) for 1 minute.
5. Discarded the supernatant without disturbing the polystyrene bead bed.
6. Added 400 μL1× Bind & Wash Buffer and vortexed for 15 seconds. Afterwards, pulse-spun, and added 100 μL1×TEX Buffer, briefly vortexed, and centrifuged at ≧10,000×g (13,000 rpm) for 1 minute.
7. Discarded the supernatant without disturbing the polystyrene bead bed.
8. Added 400 μL1× Bind & Wash Buffer and vortexed for 15 seconds. Afterwards, pulse-spun, and added 100 μL1×TEX Buffer, briefly vortexed, and centrifuged at ≧10,000×g (13,000 rpm) for 1 minute.
9. Discarded the supernatant without disturbing the polystyrene bead bed.
10. Resuspended the beads in 500 μL1× Quick Ligase Buffer. Vortexed for 15 seconds and centrifuged at ≧10,000×g (13,000 rpm) for 1 minute.
11. Discarded the supernatant without disturbing the polystyrene bead bed.
12. Resuspended the beads in 97.5 μL1× Quick Ligase Buffer
   Ligate 5mCP1A/B and 5mC-P2A/B Adapters to DNA

(SEQUENCE ID NO: 16)

5mC-P1-A CCACTACGCCTCCGCTTTCCTCTCTATGGGCAGTCGGTGAT

(SEQUENCE ID NO: 17)

5mC-P2-A CTGCCCCGGGTTCCTCATTCTCT

The top strand adapters P1-A and P2-A were synthesized with 5mC. The Nick translation step filled in bottom strand (P1-B and P2-B) with 5mC so that both the top and bottom strands of the adapters were fully 5mC protected (from bisulfite).
Used the bisulfite-SOLiD dsAdapters: dsMethyP1 adapter=5mCP1A/“regular”B and dsMethyP2 adapter=5mC-P2A/“regular”B

### Ligated the P1 and P2 Adapters to the End-Repaired DNA

1. Calculated the amount of P1 and P2 Adapters needed for the ligation reaction based on the amount of circularized DNA from “Treat the DNA with Plasmid-Safe™ ATP-Dependent DNase”.

$$For 1 µg of purified circularized DNA with an average size of$$

$$1536 ( 1500 bp insert + 36 bp internal adaptor )$$

$$\begin{matrix}X pmol \text{/} µg DNA = 1 µg DNA \times \frac{{10}^{6} pg}{1 µg} \times \frac{1 pmol}{660 pg} \times \frac{1}{1536} \\ = 1 pmol \text{/} µg DNA\end{matrix}$$

$$\begin{matrix}Y µL adaptor needed = 1 µg DNA \times \frac{1 pmol}{1 µg DNA} \times 30 \times \\ \frac{1 µL adaptor needed}{50 pmol} \\ = 0.6 µL adaptor needed\end{matrix}$$

2. Combined:

| Components | Volume (μL) |
|---|---|
| DNA-Bead Complex | 97.5 |
| P1 Adapter (ds) (50 μM) | 0.916 |
| P2 Adapter (ds) (50 μM) | 0.916 |
| Quick Ligase | 2.5 |
| Total | Variable |

3. Incubated the reaction mixture at room temperature for 15 minutes.

### Wash the DNA-Bound Streptavidin Beads

### Washed the Bead-DNA Complex

### Prepared 1×NEBuffer 2 (See Table):

| Components | Volume (μL) |
|---|---|
| NEBuffer 2, 10× | 60 |
| Nuclease-free water | 540 |
| Total | 600 |

1. Centrifuged the adapter ligation reaction at ≧10,000×g (13,000 rpm) for 1 minute.
2. Discarded the supernatant without disturbing the polystyrene bead bed.
3. Resuspended the beads in 400 μL 1× Bead Wash Buffer and vortexed for 15 seconds. Afterwards, pulse-spun, and added 100 μL 1×TEX Buffer, briefly vortexed, and centrifuged at ≧10,000×g (13,000 rpm) for 1 minute.
4. Discarded the supernatant without disturbing the polystyrene bead bed.
5. Resuspended the beads in 400 μL 1× Bind & Wash Buffer. Vortexed for 15 seconds and pulse-spun. Added 100 μL 1×TEX Buffer, briefly vortexed, and centrifuged at ≧10,000×g (13,000 rpm) for 1 minute.
6. Discarded the supernatant without disturbing the polystyrene bead bed
7. Resuspended the beads in 400 μL 1× Bind & Wash Buffer and vortexed for 15 seconds. Afterwards, pulse-spun, and added 100 μL 1×TEX Buffer, briefly vortexed, and centrifuged at ≧10,000×g (13,000 rpm) for 1 minute.
8. Discarded the supernatant without disturbing the polystyrene bead bed
9. Resuspended the beads in 500 μL 1×NEBuffer 2. Vortexed for 15 seconds and centrifuged at ≧10,000×g (13,000 rpm) for 1 minute.
10. Discarded the supernatant without disturbing the polystyrene bead bed
11. Resuspended the beads in 96 μL 1×NEBuffer 2.
   Nick-Translate the DNA with 5mC-Containing dNTPs

### Nick-Translated the DNA

This step filled-in the 5mC-protected bottom strand adapter sequence.
1. Combined:

| Components | Volume (μL) |
|---|---|
| DNA-Bead Complex | 96 |
| 5mC-dNTP Mix (100 mM, 25 mM | 2 |
| each) | |
| DNA Polymerase I (10 U/μL) | 2 |
| Total | 100 |

2. Incubated the reaction mixture at 16° C. for 30 minutes.
3. Centrifuged the nick-translation reaction at ≧10,000×g (13,000 rpm) for 1 minute.
4. Discarded the supernatant without disturbing the polystyrene bead bed.
5. Resuspended the beads in 400 μL Buffer EB (Qiagen). Vortexed for 15 seconds and pulse-spun. Add 100 μL 1×TEX Buffer, briefly vortexed, and centrifuged at ≧10,000×g (13,000 rpm) for 1 minute.
6. Discarded the supernatant without disturbing the polystyrene bead bed.
7. Suspended the beads in 500 μL of Lo-TE.
8. Optional saved 50 μL of the 500 uL nick-translated library DNA, in case a library QC needed to be run for troubleshooting purposes.

### Bisulfite Conversion

One strand of the double stranded library was eluted off the polystyrene beads with dilute NaOH. The biotinylated strand of the library was left attached to the beads. Either or both of these single stranded libraries could be bisulfite converted.

1. Freshly Prepared the Bisulfite Conversion Reagent:

| Components | Volume (μL) |
|---|---|
| Zymo CT conversion reagent | |
| (1 tube) | |
| Nuclease free water | 750 |
| M Dilution Buffer | 210 |
| Total | ~1000. |

Vortexed intermittently over 10 minutes to completely dissolve the sodium bisulfite.

### Prepared 0.1 M NaOH (User Supplied)

### Co-Processing of Bisulfite-in-Solution and Bisulfite-on-Bead

1. Centrifuged the nick translated DNA on polystyrene beads (in 500 μL of Lo-TE) at ≧10,000×g (13,000 rpm) for 1 minute.
2. Removed as much of the Lo-TE as possible, minimizing disruption of the polystyrene bead bed.
3. Added 50 μL of 0.1 M NaOH, vortexed for 15 sec and pulse-spun. Incubated for 10 minutes at room temperature to elute the non-biotinylated ssDNA library into the NaOH solution.
4. Centrifuged the beads at ≧10,000×g (13,000 rpm) for 1 minute.
5. Carefully transferred the NaOH solution (supernatant) into a MicroAmp tube.
6. Added 100 μL of the freshly prepared CT bisulfite reagent to the NaOH supernatant, mixed by pipeting up and down a couple of times, capped and incubated at 50° C. for 8 hrs in a thermalcycler.
7. Resuspended the beads in 500 μL of Lo-TE to keep as a reserve OR proceeded to step 8 to co-process (bisulfite-convert) the other strand of the library on the polystyrene beads
8. If co-processing the bisulfite-on-beads, added 50 μL of the freshly prepared CT bisulfite reagent to the polystyrene beads. Mixed the bead and bisulfite mixture by pipetting up and down a couple of times and transferred the slurry to a MicroAmp tube. Added another 50 μL of the freshly prepared CT bisulfite reagent to the original 1.5 mL Lo-Bind Tube(s) to rinse any remaining beads and transferred to the MicroAmp tube (total volume was now ˜100 μL). Capped and incubated at 50° C. for 8 hrs in a thermalcycler.

### Post Bisulfite Reaction Processing—Bisulfite-in-Solution

Required an Invitrogen cat# K310050 Purelink PCR Micro kit supplied with a desulfonation solution.

### Desalting and Desulfonation

A. Captured Bisulfite converted Library on a PureLink Column
1. Added 600 ul Purelink binding buffer (B2) to the PureLink column, and transferred the sample(s) (150 μL bisulfite reaction) into the column containing the binding buffer. Closed the cap and mixed by inverting the column several times.
2. Centrifuged at 10,000 rpm for 1 minute. Discarded the flow-through.
3. Added 600 μL wash buffer to the column, centrifuged at 10,000 rpm for 1 minute or until all the wash buffer was through the filter.

B. Desulfonation
1. Added 200 μL desulfonation buffer to the column and let stand at room temperature (20-30° C.) for 15 minutes. After the incubation, centrifuged 1 minute at 10,000 rpm. Discarded the flow-through.
2. Added 400 μL wash buffer again and centrifuged for 2 minutes at 10,000 rpm to make sure there was no trace amount of wash buffer left on the column. If it was necessary, discarded the flow-through and spun for another 1 minute at 10,000 rpm.
3. Transferred the column to a new elution tube. Added 30 μL of Lo-TE directly to the column matrix. Left at room temperature for 2 minutes and then centrifuged for 1 minute at 10,000 rpm.

### Post Bisulfite Reaction Processing—Bisulfite-on-Bead

### Desalting and Desulfonation

1. Transferred the 100 μL of the bisulfite-on-bead slurry from the microamp tube(s) into a 1.5 mL Lo-bind tube using a total of 600-800 μL of nuclease free water in portions in order to use as rinses during the transfer.
2. Centrifuged the diluted bisulfite reaction at ≧10,000×g (13,000 rpm) for 1 minute.
3. Removed as much of the supernatant without bead loss.
4. Replenished the removed supernatant with nuclease free water (up to ˜600 μL), vortexed for 15 sec, pulse-spun, added 100 μL TEX buffer, vortexed briefly and centrifuged at ≧10,000×g (13,000 rpm) for 1 minute.
5. Removed as much of the supernatant without bead loss.
6. Repeated steps 4 and 5 two times.
7. Added 500 μL of 0.1 M NaOH, vortexed for 15 sec, pulse-spun, and allowed to sit at room temperature for 15 minutes. Briefly vortexed and pulse spun a couple of times during the 15 minute wait.
8. Added 100 μL of 1×TEX Buffer, briefly vortexed, and centrifuged at ≧10,000×g (13,000 rpm) for 1 minute.
9. Discarded the supernatant without disturbing the polystyrene bead bed.
10. Added 500 μL of nuclease free water and vortexed for 15 seconds. Afterwards, pulse-spun, and added 100 μL1×TEX Buffer, briefly vortexed, and centrifuged at ≧10,000×g (13,000 rpm) for 1 minute.
11. Discarded the supernatant without disturbing the polystyrene bead bed.
12. Added 500 μL of Lo-TE and vortexed for 15 seconds. Do Not add TEX. Centrifuged at ≧10,000×g (13,000 rpm) for 1 minute.
13. Discarded the supernatant without disturbing the polystyrene bead bed.
14. Resuspended in 30 μL of Lo-TE per sample and proceeded to “Amplify the Library”.

### Amplify the Library

Both the Bisulfite-in-Solution and Bisulfite-on Beads could be processed similiarly (same volume) but the user must have ensured that the beads were suspended in solution before removing the two 2 μL aliquots. The correct number of cycles of PCR needed for optimal amplification of the bulk of the library was determined during a trial PCR.
1. Prepared a serial dilution of the bisulfite converted library as follows across one row of a PCR plate:

| 1 | 2 | 3 | 4 | 5 | 6 | 7 | 8 | 9 | 10 | 11 | 12 |
|---|---|---|---|---|---|---|---|---|---|---|---|
| UnDiluted | ½ | ¼ | ⅛ | 1/16 | 1/32 | 1/64 | 1/128 | 1/256 | 1/512 | 1/1024 | H₂O |

The serially diluted bisulfite DNA library volume was 2 μL per well. Well #1 was 2 μL of the undiluted bisulfite DNA library. Introduced 2 μL of H₂O into wells #2-12. Added a second 2 μL aliquot of the bisulfite-DNA library to the 2 μL of H₂O in well #2. Pipetted up and down to mix, and transferred 2 μL into well #3. Mixed by pipetting and transferred 2 μL into the adjacent well. Repeated this procedure until well #11, where the final 2 μL of the serial dilution was discarded. Well #12 served as the blank.
2. Prepared the master mix with Platinum PCR mix:

| | | Volume (μL) 14X (12 |
|---|---|---|
| Component | Volume (μL) 1X | wells) |
| Platinum PCR Master Mix | 22 | 308 |
| Library PCR Primer 1, 50 μM | 0.5 | 7 |
| Library PCR Primer 2, 50 μM | 0.5 | 7 |
| AmpliTaq LD 5.0 U/μL | 0.5 | 7 |
| Total | 23.5 | 329 |

3. Added 23.5 μL of the master mix to each well, bringing the total volume per well to 25.5 μL.
4. Performed 20 cycles of PCR as shown in the following table:

| Stage | Step | Temp | Time |
|---|---|---|---|
| Holding | Denature | 95° C. | 5 min |
| Cycling (20 cycles) | Denature | 95° C. | 15 sec |
| | Anneal | 62° C. | 15 sec |
| | Extend | 70° C. | 1 min |
| Holding | — | 4° C. | ∞ |

5. If library amplification was not detected in any of the wells, SOLiD sequencing was not performed.

### Confirmed Library Amplification Using Lonza FlashGel®

1. Added 1 μL 5× FlashGel® Loading Dye to 4 μL from the 100 μL PCR reaction and loaded on a 2.2% Lonza FlashGel®. Loaded FlashGel® DNA Marker (50 bp-1.5 kb or 100 bp-4 kb) in an adjacent well for reference.
2. Ran the FlashGel® for 6 minutes at 275 V.
3. Calculated the optimum number of PCR cycles that provided detectable product.

### Amplified Library

1. Performed PCR on the remaining bisulfite-converted library based on using 4 μL of library solution per each 51-μL volume PCR reaction. Dividing 56 μL by 4 μL required 14×51 μL PCR reactions. Therefore, 16× master mix was prepared (for filling the 14 wells), and 47 μL of the master mix was aliquoted into the 14 wells. The 4 μL of template solution was added last and mixed by pipetting up and down a few times.

| Component | Volume (μL) 1X | Volume (μL) 16 X |
|---|---|---|
| Platinum PCR Master Mix | 44 | 1408 |
| Library PCR Primer 1, 50 μM | 1 | 32 |
| Library PCR Primer 2, 50 μM | 1 | 32 |
| AmpliTaq LD 5.0 U/μL | 1 | 32 |
| Bisulfite Library | 4 | |
| Total | 51 | |

2. Prepared the PCR components as shown above. Vortexed to mix and then divided evenly among the required number of PCR wells.
3. Ran the PCR according to the following settings:

| Stage | Step | Temp | Time |
|---|---|---|---|
| Holding | Denature | 95° C. | 5 min |
| Cycling (TBD | Denature | 95° C. | 15 sec |
| during trial PCR) | Anneal | 62° C. | 15 sec |
| | Extend | 70° C. | 1 min |
| Holding | — | 4° C. | ∞ |

4. Pooled all of the PCR samples (from like-source, i.e. kept the bisulfite-in-solution together and the bisulfite-on-beads together when processing both) into a 1.5-mL LoBind tube.
5. If the pooled reactions were library amplification from the polystyrene beads, centrifuged at ≧10,000×g (13,000 rpm) for 1 minute.
6. Transferred the pooled supernatant off the beads into a fresh 1.5-mL LoBind tube. Re-suspended the beads in 500 μL of Lo-TE and set aside until successful Bisulfite-SOLiD sequencing was performed.
   Purified the DNA with Qiagen QIAquick® Gel Extraction Kit
1. Added 3 volumes Buffer QG and 1 volume isopropyl alcohol to the pooled PCR product. If the color of the mixture was orange or violet, added 10 μL 3 M sodium acetate, pH 5.5 and mixed. The color turned yellow. The pH required for efficient adsorption of the DNA to the membrane was ≦7.5.
2. Applied 750 μL PCR product in Buffer QG two columns.
3. Let the columns stand for 2 minutes at room temperature.
4. Centrifuged the columns at ≧10,000×g (13,000 rpm) for 1 minute and discarded the flow-through.
5. Repeated steps 2 and 4 until the entire sample had been loaded onto the columns. Placed the MinElute® columns back into the same collection tube.
6. Added 750 μL Buffer PE to wash the columns.
7. Centrifuged the columns at ≧10,000×g (13,000 rpm) for 2 minutes. Discarded the flow-through. Repeat to remove residual wash buffer.
8. Air-dried the columns for 2 minutes to evaporate any residual alcohol. Transferred the columns to clean 1.5-mL LoBind tube(s).
9. Added 30 μL Buffer EB to the column(s) to elute the DNA and let the columns stand for 2 minutes.
10. Centrifuged the column(s) at ≧10,000×g (13,000 rpm) for 1 minute.
11. If necessary, pooled the eluted DNA.

### Gel-Purify the Library

Size-Selecdt the DNA Fragments with an Agarose Gel
1. To the 30 μL of QiaQuick purified library was added 3 μL of 10×PCR buffer and 6 μL of “5× Gel Pilot Loading Dye” resulting in a total volume of 39 μL. This volume required 2 wells of the BioRad precast gel.
2. Loaded 2 μL TrackIt™ 25 by Ladder. The brightest band for this size ladder was 125 bp. Loaded ˜20 μL dye-mixed sample per well. At least one lane was present between the ladder well and the sample wells to avoid contamination of the sample with ladder.
3. Ran the gel at 120 V until the marker was close to the edge of the gel.
4. If needed, stained the gel in 50 to 100 mL 1×TAE or 1×TBE Buffer with 8 μL ethidium bromide (10 mg/mL) for 5 minutes.
5. Destained the gel in nuclease-free water twice for 2 minutes each time and visualized the gel on a UV transilluminator.
6. Excised the entire band which had an average size ranging from 200 to 300 by using a clean razor blade.

### Eluted the DNA Using Qiagen QIAquick® Gel Extraction Kit

1. Weighed the gel slice(s) in a 15-mL polypropylene conical colorless tube.
2. Added 6 volumes Buffer QG to 1 volume of gel.
3. Dissolved the gel slice by vortexing at room temperature until the gel slice had dissolved completely (˜5 minutes).
4. If the color of the mixture was yellow, proceeded to step 5. If the color of the mixture was orange or violet, added 10 μL 3 M sodium acetate, pH 5.5 and mixed. The pH required for efficient adsorption of the DNA to the membrane was ≦7.5.
5. Added one gel volume of isopropyl alcohol to the sample and mixed by inverting the tube several times.
6. Applied about 700 μL sample to the column(s). The maximum amount of gel that could be applied to a MinElute® column was 400 mg. Used more columns as necessary.
7. Let the column(s) stand for 2 minutes at room temperature.
8. Centrifuged the column(s) at ≧10,000×g (13,000 rpm) for 1 minute and discarded the flow-through.
9. Repeated steps 6 and 8 until the entire sample had been loaded onto the column(s). Placed the MinElute® column(s) back into the same collection tube.
10. Added 750 μL Buffer PE to wash the column(s).
11. Centrifuged the column(s) at ≧10,000×g (13,000 rpm) for 2 minutes. Discarded the flow-through. Repeated to remove residual wash buffer.
12. Air-dried the column(s) for 2 minutes to evaporate any residual alcohol. Transferred the column(s) to clean 1.5-mL LoBind tube(s).
13. Added 30 μL Buffer EB to the column(s) to elute the DNA and let the column(s) stand for 2 minutes.
14. Centrifuged the column(s) at ≧10,000×g (13,000 rpm) for 1 minute.
15. If necessary, pooled the eluted DNA in a 1.5-mL LoBind tube.
   Quantitate the library by Qbit and BioAnalyzer
   Qbit quantitation of the bisulfite-on-bead library was 1.2 ng/μL
   Qbit quantitation of the bisulfite-in-solution library was 2.4 ng/μL
   Quantitated the library by performing quantitative PCR (qPCR)

| Quantitation method | Sensitivity |
|---|---|
| Lonza 2.2% FlashGel ® with FlashGel ® | 3 ng/μL |
| QuantLadder | |
| Invitrogen Qubit ™ | 200 pg/μL |
| Agilent Bioanalyzer DNA 1000 Assay | 100 pg/μL |

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### EQUIVALENTS

The foregoing description and Examples detail certain specific embodiments of the present teachings and describes the best mode contemplated by the inventors. It will be appreciated, however, that no matter how detailed the foregoing can appear in text, the present teachings can be practiced in many ways and the invention should be construed in accordance with the appended claims and any equivalents thereof.

What is claimed is:

1. A method of analyzing the methylation state of genomic DNA, comprising:
fragmenting a genomic DNA sample, whereby genomic DNA fragments are produced;
circularizing a genomic DNA fragment to produce a double-stranded circular DNA comprising a nick on at least one strand of the double-stranded circular DNA;
linearizing the circular DNA;
adding a nick translation enzyme in the presence of methylation conversion agent resistant nucleotide triphosphates, whereby a partially methylation conversion agent resistant polynucleotide is generated, wherein the partially methylation conversion agent resistant polynucleotide has a tag region that is methylation conversion agent resistant and a tag region that is not methylation conversion agent resistant.

2. The method of claim 1, further comprising exposing the partially methylation conversion agent resistant polynucleotide to a methylation conversion agent, whereby a conversion agent treated polynucleotide is produced.

3. The method of claim 2, wherein the polynucleotide exposed to the methylation conversion agent is amplified to produce an amplicon.

4. The method of claim 3, further comprising sequencing a region of the amplicon that is derived from the tag region that is methylation conversion agent resistant, and sequencing a region of the amplicon that is derived from the tag region that is not methylation conversion agent resistant.

5. The method of claim 1, wherein the circular DNA comprises a specific binding pair member.

6. The method of claim 5, wherein the specific binding pair member is biotin.

7. The method of claim 5, further comprising the step of attaching adapters to the ends of the partially methylation conversion agent resistant polynucleotide to produce an adapter modified polynucleotide.

8. The method of claims 7, wherein the adapters are double-stranded, and wherein at least one of the stands contains methylation conversion resistant nucleotides and at least one of the strands comprises a first primer binding site sequence.

9. The method of 8, further comprising exposing the adapter modified polynucleotide to a nick translation enzyme and a set of dNTPS.

10. The method of claim 9, further comprising:
exposing the adapter modified polynucleotide to a cognate receptor of the specific binding pair member;
denaturing the adapter modified polynucleotide; and
exposing strands of the adapter modified polynucleotide that are not bound to the cognate receptor to a methylation conversion agent, whereby converted stands are produced.

11. The method of claim 10, further comprising preferentially amplifying the converted strands.

12. The method of claim 11, wherein the preferential amplification introduces a second primer binding site on one end, but not the other end of the preferential amplification product.

13. The method of claim 9, further comprising:
exposing the adapter modified polynucleotide to a cognate receptor of the specific binding pair member;
denaturing the adapter modified polynucleotide; and
separating strands of the adapter modified polynucleotide that are not bound to the cognate receptor from strands of the adapter modified polynucleotide that are bound to the cognate receptor.

14. The method of claim 13, wherein the cognate receptor is bound to a solid support.

15. The method of claim 14, wherein the cognate receptor comprises streptavidin bound to non-magnetic polystyrene beads.

16. The method of claim 13, further comprising:
exposing at least one of the strands of the adapter modified polynucleotide that are not bound to the cognate receptor and strands of the adapter modified polynucleotide that are bound to the cognate receptor to a methylation conversion agent, whereby converted stands are produced.

17. The method of claim 16, further comprising preferentially amplifying the converted strands.

18. The method of claim 17, wherein the preferential amplification introduces a second primer binding site on one end of the preferential amplification product but not on the other end of the preferential amplification product.

19. A method of analyzing the methylation state of genomic DNA, comprising:
fragmenting a genomic DNA sample, whereby genomic DNA fragments are produced;
forming a first tag sequence and a second tag sequence, wherein the first tag sequence and the second tag sequence are derived from a single genomic DNA fragment;
wherein the first tag sequence has been converted by a methylation conversion agent and the second tag sequence has not been converted by a methylation conversion agent.

20. The method of claim 19, wherein the first tag sequence and the second tag sequence are present on a single polynucleotide molecule.

21. The method of claim 20, further comprising amplifying the single polynucleotide molecule to produce an amplicon.

22. The method of claim 21, wherein the amplification is clonal amplification.

23. The method of claim 21, wherein the clonal amplification is solid phase amplification.

24. The method of claim 22, wherein the clonal amplification is emulsion PCR.

25. A polynucleotide construction comprising a first tag sequence and a second tag sequence, wherein the first tag sequence and the second tag sequence are derived from a single fragment of genomic DNA, wherein the first tag comprises methylation conversion resistant nucleotide that have been incorporated into the construction by an in vitro reaction and the second tag does not comprise methylation conversion resistant nucleotide that have been incorporated into the construction by an in vitro reaction.

26. The polynucleotide construction of claim 25, further comprising an internal adapter located between the first tag and the second tag.

27. The polynucleotide construction of claim 26, wherein the internal adapter comprises a specific binding pair member.

28. The polynucleotide construction of claim 27, further comprising primer binding sequences located in functional proximity to the first tag sequence and the second tag sequence, wherein amplification primers binding to the priming sites can amplify both the first and the second tag sequences.

29. An adapter comprising a first strand having methylation conversion resistant nucleotides and a second strand complementary to the first strand, wherein the second strand optionally contains methylation conversion resistant nucleotides.

30. A kit comprising an adapter of claim 29 and oligonucleotide primers specific for a strand of the adapter.

31. A method of matching a DNA sequence to a genomic sequence database, said method comprising:
comparing a data record comprising (1) a first tag sequence that corresponds to a DNA sequence that has not been modified by a methylation conversion agent and (2) a second tag sequence that corresponds to a DNA sequence that may have been modified by a methylation conversion agent, with DNA sequence information in the genomic database.

32. The method of claim 31, wherein comparing the data record uses a value indicative of the approximate distance in the genome between the first tag sequence and the second tag sequence.

33. The method of claim 32, further comprising detecting a first nucleic acid sequence in the genomic sequence database that corresponds to the first tag sequence and detecting a second nucleic acid sequence in the genomic sequence database that corresponds to the second tag sequence.

34. The method of claim 33, further comprising comparing the second tag sequence with the corresponding genomic reference sequence to detect sequence differences indicative of methylation of a region of genomic DNA from which the second tag sequence was derived.

35. The method of claim 33, further comprising:
comparing a plurality of second tag sequence with a corresponding genomic reference sequence;
determining a value or set of values indicative of the degree of methylation of a base or bases in the second tag sequence; and
displaying the value or set of values indicative of the degree of methylation of a base or bases in the second tag sequence.

36. A method of amplifying polynucleotides converted by a methylation conversion agent, comprising;
providing a polynucleotide fragment having two termini;
ligating a primer-adapter to both of the termini, wherein the primer-adapter is a double-stranded polynucleotide having a first stand and second strand complementary to the first strand, wherein the first strand comprises methylation conversion resistant nucleotides and the second strand optionally comprises methylation conversion resistant nucleotides, whereby an adapter modified polynucleotide is produced;
exposing the adapter-modified polynucleotide to a methylation conversion reagent, whereby a converted adapter modified polynucleotide is produced; and
amplifying the converted adapter modified polynucleotide, wherein amplifying the converted adapter modified polynucleotide uses primers specific for sequences in the second strand of the adapter.

37. The method of claim 36, further comprising:
denaturing the adapter modified polynucleotide to produce separated strands;
enriching one of the separated strands; and
performing the amplification step on the enriched strand.

38. A method of analyzing the methylation state of a genomic DNA sample, said method comprising:
mixing a DNA sample with formamide, whereby a sample mixture is formed;
heating the sample mixture at temperature sufficient to denature the genomic DNA; and
adding a bisulfite salt to the sample mixture.

39. The method of claim 38, wherein the formamide concentration in the sample mixture prior to the addition of the bisulfite salt is a least 50%.

40. The method of claim 39, wherein the formamide concentration in the sample mixture prior to the addition of the bisulfite salt is a least 75%.

41. The method of claim 40, wherein the formamide concentration in the sample mixture prior to the addition of the bisulfite salt is a least 90%.

42. The method of claim 41, wherein the formamide concentration in the sample mixture prior to the addition of the bisulfite salt is a least 95%.

43. The method of claim 38, wherein the DNA sample is present in a gel matrix.

44. The method of claim 43, wherein the gel matrix comprise polyacrylamide.

45. The method of claim 38, wherein the DNA sample is derived from a paraffin embedded sample.

46. The method of claim 43, further comprising the step of amplifying the DNA sample in the gel matrix, wherein the amplification occurs within the matrix and the amplification occurs after the bisulfite has been added.

47. A method of analyzing the methylation state of a polynucleotide, comprising:
providing a polynucleotide fragment having two termini;
ligating a primer-adapter to both of the termini;
circularizing the adapter-modified polynucleotide with an internal adapter to produce a double-stranded circular polynucleotide comprising a nick on one strand of the circular polynucleotide, wherein the internal adapter comprises a specific binding moiety;
nick-translating the circular polynucleotide;
capturing the strand comprising the specific binding moiety with a cognate specific binding moiety on a solid support;
separating the captured strand and the non-captured strand; and
exposing at least one of the captured strand and the non-captured strand to a methylation conversion reagent, whereby at least one converted strand is produced; and
sequencing the at least one converted strand.

48. The method of claim 47, wherein the specific binding moiety comprises biotin.

49. The method of claim 48, wherein the cognate specific binding moiety is chosen from avidin and streptavidin.

50. The method of claim 49, wherein the solid support comprises a non-magnetic polystyrene bead.


SEQUENCE LISTING (28 sequences)

SEQ ID NO: 1 - DNA, 20 bp, Artificial Sequence
  Description of Artificial Sequence Synthetic oligonucleotide
ggccaaggcggatgtacggt

SEQ ID NO: 2 - DNA, 41 bp, Artificial Sequence
  Description of Artificial Sequence Synthetic oligonucleotide
ccactacgcctccgctttcctctctatgggcagtcggtgat

SEQ ID NO: 3 - DNA, 53 bp, Artificial Sequence
  Description of Artificial Sequence Synthetic primer
ctgccccgggttcctcattctaaccactacacctccactttcctctctataaa

SEQ ID NO: 4 - DNA, 41 bp, Artificial Sequence; identical to SEQ ID NO: 2
  Description of Artificial Sequence Synthetic oligonucleotide

SEQ ID NO: 5 - DNA, 43 bp, Artificial Sequence
  Description of Artificial Sequence Synthetic oligonucleotide
atcaccgactgcccatagagaggaaagcggaggcgtagtggtt

SEQ ID NO: 6 - DNA, 23 bp, Artificial Sequence
  Description of Artificial Sequence Synthetic oligonucleotide
ctgccccgggttcctcattctct

SEQ ID NO: 7 - DNA, 25 bp, Artificial Sequence
  Description of Artificial Sequence Synthetic oligonucleotide
agagaatgaggaacccggggcagtt

SEQ ID NO: 8 - DNA, 28 bp, Artificial Sequence
  Description of Artificial Sequence Synthetic primer
ccactacgcctccgctttcctctctatg

SEQ ID NO: 9 - DNA, 21 bp, Artificial Sequence
  Description of Artificial Sequence Synthetic primer
ctgccccgggttcctcattct

SEQ ID NO: 10 - DNA, 22 bp, Artificial Sequence
  Description of Artificial Sequence Synthetic primer
cgcctccgctttcctctctatg

SEQ ID NO: 11 - DNA, 21 bp, Artificial Sequence
  Description of Artificial Sequence Synthetic primer
ctgccccgggttcctcattct

SEQ ID NO: 12 - DNA, 22 bp, Artificial Sequence
  Description of Artificial Sequence Synthetic primer
cgcctccgctttcctctctatg

SEQ ID NO: 13 - DNA, 7 bp, Artificial Sequence
  Description of Artificial Sequence Synthetic oligonucleotide
acagcag

SEQ ID NO: 14 - DNA, 9 bp, Artificial Sequence
  Description of Artificial Sequence Synthetic oligonucleotide
ctgctgtac

SEQ ID NO: 15 - DNA, 20 bp, Artificial Sequence
  Description of Artificial Sequence Synthetic oligonucleotide
cgtacatccgccttggccgt

SEQ ID NO: 16 - DNA, 41 bp, Artificial Sequence; identical to SEQ ID NO: 2
  Description of Artificial Sequence Synthetic oligonucleotide

SEQ ID NO: 17 - DNA, 23 bp, Artificial Sequence
  Description of Artificial Sequence Synthetic oligonucleotide
ctgccccgggttcctcattctct

SEQ ID NO: 18 - DNA, 36 bp, Artificial Sequence
  Description of Artificial Sequence Synthetic oligonucleotide
ctgctgtaccgtacatccgccttggccgtacagcag

SEQ ID NO: 19 - DNA, 36 bp, Artificial Sequence
  Description of Artificial Sequence Synthetic oligonucleotide
ctgctgtacggccaaggcggatgtacggtacagcag

SEQ ID NO: 20 - DNA, 13 bp, Artificial Sequence
  Description of Artificial Sequence Synthetic oligonucleotide
tacggtacagcag

SEQ ID NO: 21 - DNA, 36 bp, Artificial Sequence
  Description of Artificial Sequence Synthetic oligonucleotide
ttgttgtatggccaaggcggatgtacggtacagcag

SEQ ID NO: 22 - DNA, 41 bp, Artificial Sequence
  Description of Artificial Sequence Synthetic oligonucleotide
atcaccgactgcccatagagaggaaagcggaggcgtagtgg

SEQ ID NO: 23 - DNA, 41 bp, Artificial Sequence
  Description of Artificial Sequence Synthetic oligonucleotide
attattgattgtttatagagaggaaagtggaggtgtagtgg

SEQ ID NO: 24 - DNA, 30 bp, Artificial Sequence
  Description of Artificial Sequence Synthetic oligonucleotide
ccactacacctccactttcctctctataaa

SEQ ID NO: 25 - DNA, 64 bp, Artificial Sequence
  Description of Artificial Sequence Synthetic oligonucleotide
ctgccccgggttcctcattctctccactacacctccactttcctctctataaacaatcaataat

SEQ ID NO: 26 - DNA, 64 bp, Artificial Sequence
  Description of Artificial Sequence Synthetic oligonucleotide
attattgattgtttatagagaggaaagtggaggtgtagtggttagaatgaggaacccggggcag

SEQ ID NO: 27 - DNA, 28 bp, Artificial Sequence
  Description of Artificial Sequence Synthetic oligonucleotide
ccactacgcctccgctttcctctctatg

SEQ ID NO: 28 - DNA, 53 bp, Artificial Sequence
  Description of Artificial Sequence Synthetic oligonucleotide
ctgccccgggttcctcattctctccactacacctccactttcctctctataaa